(19) United States
(12) Patent Application Publication
(10) Pub. No.: US 2024/0041605 A1
(43) Pub. Date: Feb. 8, 2024

(54) ORTHOPEDIC IMPLANT WITH POROUS STRUCTURE HAVING VARYING COEFFICIENT OF FRICTION WITH BONE

(71) Applicant: BioMedtrix, LLC, Whippany, NJ (US)

(72) Inventors: Christopher Preucil, Ketchum, ID (US); Gregory T. Van Der Meulen, Hailey, ID (US); Christopher G. Sidebotham, Mendham, NJ (US)

(73) Assignee: BioMedtrix, LLC, Whippany, NJ (US)

(21) Appl. No.: 18/485,626

(22) Filed: Oct. 12, 2023

Related U.S. Application Data
(63) Continuation of application No. PCT/US2022/024836, filed on Apr. 14, 2022.
(60) Provisional application No. 63/175,018, filed on Apr. 14, 2021.

(51) Int. Cl. A61F 2/30; A61F 2/36

(52) U.S. Cl. CPC A61F 2/30767; A61F 2/3662; A61F 2002/3093; A61F 2002/3092; A61F 2002/30024

(57) ABSTRACT

An orthopedic implant has an implant body defining a longitudinal axis extending in a direction of implantation of the orthopedic implant, the implant body having a first end portion and a second end portion. A porous structure extends circumferentially around the implant body and has a first circumferentially-extending zone exhibiting a first coefficient of friction with bone tissue and a second circumferentially-extending zone exhibiting a second coefficient of friction with bone tissue, wherein the second coefficient of friction is greater than the first coefficient of friction. The first circumferentially-extending zone of the porous structure is offset from the second circumferentially-extending zone along the longitudinal axis such that during implantation of the orthopedic implant, the first circumferentially-extending zone of the porous structure contacts bone before the second circumferentially-extending zone of the porous structure.

### CROSS REFERENCE TO RELATED APPLICATIONS

The present application is a continuation of International Application No. PCT/US2022/024836, filed Apr. 14, 2022, which claims the benefit of U.S. Provisional Application No. 63/175,018, filed on Apr. 14, 2021, each of which is incorporated herein by reference in its entirety.

### FIELD

The present disclosure pertains to orthopedic implants including porous structures with varying surface roughness.

### BACKGROUND

Certain orthopedic implants, such as prosthetic joint components used in joint replacement procedures, can be secured in the appropriate bone with cement, or by cementless fixation. In cementless fixation, the implant typically includes a porous structure that engages the native tissue when the implant is received in the prepared bone. For example, in a total hip replacement (hip arthroplasty) procedure, the acetabular cup can include a porous structure that engages the native tissue of the acetabular socket. The preparation is typically undersized relative to the size of the implant to facilitate an interference fit between the implant and the native tissue. The porous structure of the implant promotes stability by limiting movement of the implant relative to the bone to facilitate bone ingrowth. However, such porous structures typically have a uniformly high coefficient of friction with bone tissue, and strongly engage the bone upon surface contact. Thus, attempting to reposition such an implant after initial placement typically destroys the preparation by damaging the surrounding tissue, resulting in the need to remove the implant (often causing further tissue damage) and prepare the implantation site again. Accordingly, there exists a need for improved orthopedic implants configured for cementless fixation that permit in situ repositioning of the implant during implantation.

### SUMMARY

The present disclosure pertains to orthopedic implants including porous structures with varying surface roughness. In a representative example, an orthopedic implant comprises an implant body defining a longitudinal axis extending in a direction of implantation of the orthopedic implant, the implant body having a first end portion and a second end portion; and a porous structure extending circumferentially around the implant body, the porous structure comprising a first circumferentially-extending zone exhibiting a first coefficient of friction with bone tissue and a second circumferentially-extending zone exhibiting a second coefficient of friction with bone tissue, wherein the second coefficient of friction is greater than the first coefficient of friction; and wherein the first circumferentially-extending zone of the porous structure is offset from the second circumferentially-extending zone along the longitudinal axis such that during implantation of the orthopedic implant, the first circumferentially-extending zone of the porous structure contacts bone before the second circumferentially-extending zone of the porous structure.

In another representative embodiment, an orthopedic implant comprises an implant body defining a longitudinal axis extending in a direction of implantation of the orthopedic implant, the implant body having a first end portion and a second end portion. The implant body has a thickness dimension defined perpendicular to the longitudinal axis, the thickness dimension of the implant body increasing in a direction along the longitudinal axis from the first end portion to the second end portion. The implant body comprises a porous structure extending along the longitudinal axis, the porous structure comprising a plurality of struts arranged in a three-dimensional arrangement, the porous structure comprising a transition zone in which the porous structure gradually develops from a solid bulk material of the implant body over a selected distance.

In another representative embodiment, an orthopedic implant comprises an implant body defining a longitudinal axis extending in a direction of implantation of the orthopedic implant, the implant body having a first end portion and a second end portion; and a porous structure extending circumferentially around the implant body, the porous structure comprising a first circumferentially-extending zone exhibiting a first coefficient of friction with bone tissue and a second circumferentially-extending zone exhibiting a second coefficient of friction with bone tissue, wherein the second coefficient of friction is greater than the first coefficient of friction.

In another representative embodiment, an orthopedic implant comprises an implant body defining a longitudinal axis extending in a direction of implantation of the orthopedic implant, the implant body having a first end portion and a second end portion; the implant body having a thickness dimension defined perpendicular to the longitudinal axis, the thickness dimension of the implant body increasing in a direction along the longitudinal axis from the first end portion to the second end portion; and the implant body comprising a porous structure extending along the longitudinal axis, the porous structure exhibiting a coefficient of friction with bone tissue that decreases along the porous structure in a direction of implantation of the orthopedic implant.

In another representative embodiment, an orthopedic implant comprises an implant body comprising a solid substrate and a porous bone-contacting structure on the solid substrate, the porous bone-contacting structure comprising a plurality of angled strut members extending outwardly from the solid substrate of the implant body; wherein a first portion of the strut members of the porous bone-contacting structure extend from the solid substrate by a specified distance or less; and wherein a second portion of the strut members of the porous bone-contacting structure extend beyond the specified distance and comprise pointed end portions.

The foregoing and other objects, features, and advantages of the disclosed technology will become more apparent from the following detailed description, which proceeds with reference to the accompanying figures.

### BRIEF DESCRIPTION OF THE DRAWINGS

FIG. 1 is a side elevation view of representative embodiments of orthopedic implants for total hip replacement in an exploded state.

FIG. 2 illustrates the orthopedic implants of FIG. 1 in an assembled state.

FIG. 3 is a micrograph illustrating a three-dimensional porous structure of the acetabular cup of FIG. 2, according to one embodiment.

FIG. 4 is a side elevation view of an acetabular cup including a three-dimensional porous structure having zones of different surface roughness, according to one embodiment.

FIG. 5 is a cross-sectional view of the acetabular cup of FIG. 4 taken along line 5-5 of FIG. 4.

FIG. 6 is a schematic side view of a portion of a three-dimensional porous structure illustrating strut members with differently shaped end portions, according to one embodiment.

FIG. 7 is a micrograph illustrating end portions of strut members of a portion of a three-dimensional porous structure having a relatively low surface roughness.

FIG. 8 is a micrograph illustrating end portions of strut members of a portion of a three-dimensional porous structure having a relatively high surface roughness.

FIG. 9 is a side elevation view of an acetabular cup illustrating zones of different surface roughness, according to one embodiment.

FIGS. 10-12 schematically illustrate a representative method of implanting the acetabular cup of FIG. 9.

FIG. 13 is a side elevation view of a femoral stem prosthesis including a three-dimensional porous structure with zones of different surface roughness, according to one embodiment.

FIG. 14 illustrates the acetabular cup of FIG. 4 and the femoral stem prosthesis of FIG. 13 implanted to form a prosthetic canine hip joint.

FIG. 15 is a perspective view of a canine elbow implant including three-dimensional porous structures, according to one embodiment.

FIG. 16 is a perspective view of a canine ankle implant including three-dimensional porous structures, according to one embodiment.

FIG. 17 is a perspective view of a tibial implant for canine stifle replacement including a three-dimensional porous structure, according to one embodiment.

FIG. 18 is a perspective view of a femoral implant for canine stifle replacement including three-dimensional porous structures with zones of different surface roughness, according to one embodiment.

FIGS. 19A and 19B are a plan view and a side elevation view, respectively, of a test member with a test material formed thereon.

FIG. 20 is a schematic diagram illustrating a representative embodiment of a test apparatus for determining the coefficient of friction of the test material formed on the test member of FIGS. 19A and 19B with simulated cancellous bone material.

FIG. 21 is a diagram illustrating the forces acting on the test member of FIGS. 19A and 19B disposed on the test apparatus of FIG. 20.

FIGS. 22A-22D are micrographs of various three-dimensional porous structures analyzed in a test.

FIG. 23 is a bar chart illustrating the results of a test performed with the porous structures shown in FIGS. 22A-22D using the test apparatus of FIG. 20.

FIG. 24 is a magnified view of another example of a porous lattice structure including rounded strut ends and relatively uniform strut diameters.

FIG. 25 is a magnified view of another example of a porous lattice structure in which the struts include prominences and recesses for increased friction.

FIG. 26 is a magnified view of an example of a transition zone of a porous structure.

FIG. 27 is a side elevation view of another example of a femoral stem prosthesis including a porous structure with a plurality of zones having different coefficients of friction with bone.

FIG. 28 is a side elevation view of another example of a femoral stem prosthesis including a porous structure with two transition zones.

FIG. 29 is a perspective view of another example of an acetabular cup including a porous conformal lattice structure with zones having different coefficients of friction with bone.

FIG. 30 is a magnified view of a portion of the transition zone of the porous structure of FIG. 29.

FIG. 31 is a magnified view of a portion of a high friction zone of the porous structure of FIG. 29.

### DETAILED DESCRIPTION

Orthopedic implants for use in joint replacement procedures are typically configured to be held in place using bone cement or, alternatively, to be press-fitted into the bone and held in place by frictional engagement with the surrounding tissue. Such “cementless” implants can include one or a plurality of regions comprising a porous structure configured to exhibit a high coefficient of friction with bone or other native tissue, and to promote bone ingrowth for long term stabilization of the implant. In certain examples, such porous structures can comprise a three-dimensional scaffold and/or lattice comprising a plurality of angled, interconnected members, struts and/or rods, referred to hereinafter as strut members. The strut members can be arranged to define openings and/or pores between them, and throughout the extent of the three-dimensional porous structure. In certain embodiments, the three-dimensional porous structure can be formed using three-dimensional printing and/or additive manufacturing techniques.

In certain examples, the surface roughness of the three-dimensional porous structure can be varied at different locations on a porous structure of an implant to vary its adhesion with bone or other native tissue. For example, in certain embodiments the porous structure of an implant can be configured such that the surface of the porous structure comprises a plurality of regions or zones having different surface roughness values, and thereby exhibiting different coefficients of friction with bone or other native tissue. In certain embodiments, the porous structure can be configured such that its surface roughness, and thereby its coefficient of friction with bone or other native tissue, varies continuously along the surface of the porous structure in a particular direction, such as in the direction of implantation.

In certain embodiments, the strut members of the porous structure can be formed on a solid substrate of the implant body, and can extend outwardly from the solid substrate surface. In certain embodiments, the surface roughness of the three-dimensional porous structure can be at least partially determined based on end conditions and/or end structures of the strut members. For example, in certain embodiments the surface roughness of the three-dimensional porous structure can be at least partially determined by variation in length of strut members in the porous structure relative to a reference line, a reference plane, or a reference surface of the implant located a specified distance from the solid substrate surface. For example, the surface roughness can be varied by varying one or more of the following parameters: (a) the number of strut members that extend beyond, or end short of, the reference line/plane/surface; (b) the distance by which strut members extend beyond, or end short of, the reference line/plane/surface; and/or (c) the shape and/or dimensions (e.g., width, diameter, taper, angle, etc.) of the end portions of the strut members.

Varying the parameters above, or combinations thereof, can result in variations in the coefficient of friction of the surface of the porous structure with bone or other native tissue. This can allow certain porous structures of an implant, or portions of the surfaces of such porous structures, to exhibit a higher or lower coefficient of friction than others. In a representative embodiment, the strut members of a high-friction zone of a porous structure can have angular, irregular, and/or pointed end portions, and can end at various heights, thereby increasing the surface roughness of the zone. Conversely, the strut members of a low-friction zone of the porous structure can have rounded end portions, and/or can have heights that are the same or substantially the same as each other, thereby reducing the surface roughness of the zone.

Thus, according to the embodiments described herein, the surface of a porous structure of an implant can be configured such that region(s) of the porous structure that contact native tissue early in the implantation process can exhibit a relatively lower coefficient of friction than region(s) that contact the native tissue subsequently. For example, the portion(s) of a porous structure of an implant that contact native tissue only as the implant nears its final position can exhibit a relatively higher coefficient of friction than portions that contact tissue initially. This can be particularly useful, for example, when adjustment of the position and/or orientation of an implant relative to the bone may be indicated mid-placement, before final positioning of the implant. For example, with lower friction portions of the surface of the porous structure in contact with the native tissue, the implant can be rotated, moved proximally or distally, etc., more easily and with reduced damage to the tissue of the prepared implantation site. In certain embodiments, although different portions of the surface can have relatively higher or relatively lower surface roughness, the underlying three-dimensional porous structure can be the same, facilitating bone growth into all regions of the porous structure regardless of surface roughness and frictional engagement with the surrounding bone.

The porous structure embodiments described herein can also be applicable to any of a variety of implants for different orthopedic procedures, including hip arthroplasty, knee replacement, ankle replacement, elbow replacement, etc. For purposes of illustration, the orthopedic implants shown and described herein are configured for use in canine orthopedic procedures. However, the specific examples provided herein are not intended to be limiting, and the porous structure embodiments of the present disclosure can be adapted for use on orthopedic implants for a wide variety of human and/or veterinary orthopedic procedures.

### Example 1: Acetabular Cup and Femoral Stem Prosthesis with Porous Structures Having Varying Coefficients of Friction with Bone

In a total hip replacement procedure (also known as hip arthroplasty), the articular surfaces of the native hip joint are replaced with prosthetic implants that are secured to the pelvis and the femur to form a ball-and-socket joint. The prosthetic joint assembly typically includes a femoral hip step that is inserted into the canal of the femur, a femoral head typically including a spherical bearing or ball coupled to the femoral hip stem, and an acetabular cup that is inserted into the acetabulum of the pelvis and configured to receive the prosthetic femoral head. In a typical procedure, the femur is separated from the acetabulum, and the native femoral head is removed from the femur. The acetabulum is prepared using a multi-step reaming process to remove tissue and expose healthy bone, typically with a reamer that is smaller than the acetabular cup diameter. The acetabular cup implant is secured in the prepared acetabulum. In certain examples, the cup may be press-fitted into place (e.g., by impact), and/or may be cemented into the socket. The femur is axially reamed to create an opening into which the femoral stem prosthesis can be inserted. The femoral stem prosthesis can also be press-fitted and/or cemented into place, depending upon the particular indication. The prosthetic femoral head can be attached to the femoral stem prosthesis, and the femur can be maneuvered into place such that the prosthetic femoral head is received in the acetabular cup to form a prosthetic hip joint.

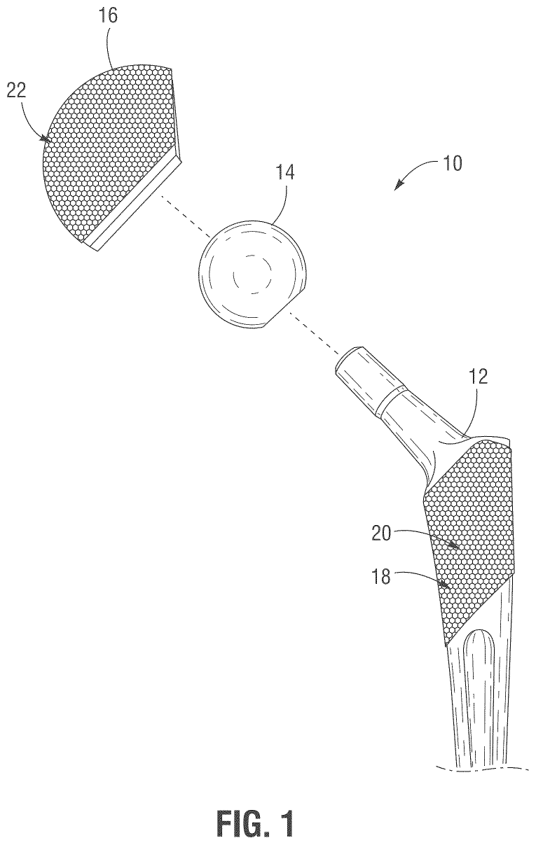

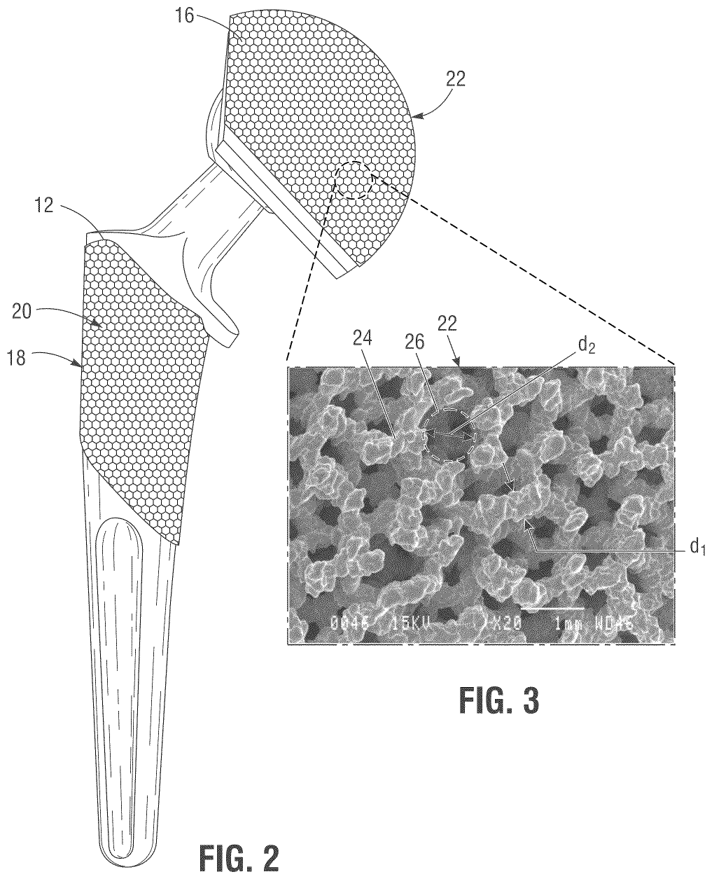

FIG. 1 illustrates an exemplary prosthetic implant assembly 10 for hip arthroplasty including a femoral stem prosthesis 12, a prosthetic femoral head 14, and an acetabular cup 16. FIG. 2 illustrates the components in the assembled state in which the prosthetic femoral head 14 is coupled to the femoral stem prosthesis 12 and received in the acetabular cup 16.

In the embodiment illustrated in FIGS. 1 and 2, the femoral stem prosthesis 12 and the acetabular cup 16 are configured primarily for cementless fixation in the respective native bones. Accordingly, the femoral stem prosthesis 12 includes a region or zone 18 near its proximal end comprising a porous bone-contacting structure 20 configured to engage the tissue of the native femoral canal. The acetabular cup 16 includes a similar porous structure 22 on its exterior surface configured to engage the tissue of the acetabular socket.

In certain examples, the porous structure can comprise a three-dimensional scaffold and/or lattice structure/arrangement comprising a plurality of angled, interconnected strut members. The strut members can be arranged to define openings and/or pores between them. For example, FIG. 3 illustrates a representative example of a three-dimensional, porous lattice structure 22 of the acetabular cup 16. The lattice structure can comprise a plurality of angled strut members 24 defining pores, openings, and/or voids 26 between them. In certain embodiments, the strut members 24 can have a specified diameter d₁, and the pores 26 can have a specified diameter d₂. The diameters d₁ and d₂ can be selected according to any of various factors, such as the type of implant, the implant material, the type of tissue at the interface with the implant, the species and/or size of the patient, etc. In certain embodiments, the three-dimensional porous structure can be formed by three-dimensional printing and/or additive manufacturing techniques. In certain embodiments, the diameter d₁ can be 0.1 mm to 0.6 mm, such as 0.2 mm to 0.5 mm, 0.6 mm or less, 0.5 mm or less, etc. In certain embodiments, the diameter d₂ can be 0.1 mm to 2 mm, such as 0.1 mm to 1.5 mm, 0.5 mm to 2 mm, 0.5 mm to 1.5 mm, 0.1 mm to 1 mm, etc.

As noted above, in certain embodiments the porous structure can be configured such that it exhibits a coefficient of friction with bone tissue that varies along the surface of the porous structure. Such variation can be continuous along the surface of the porous structure in a particular direction (e.g., in the direction of implantation), or in discrete regions or zones exhibiting different coefficients of friction with bone tissue.

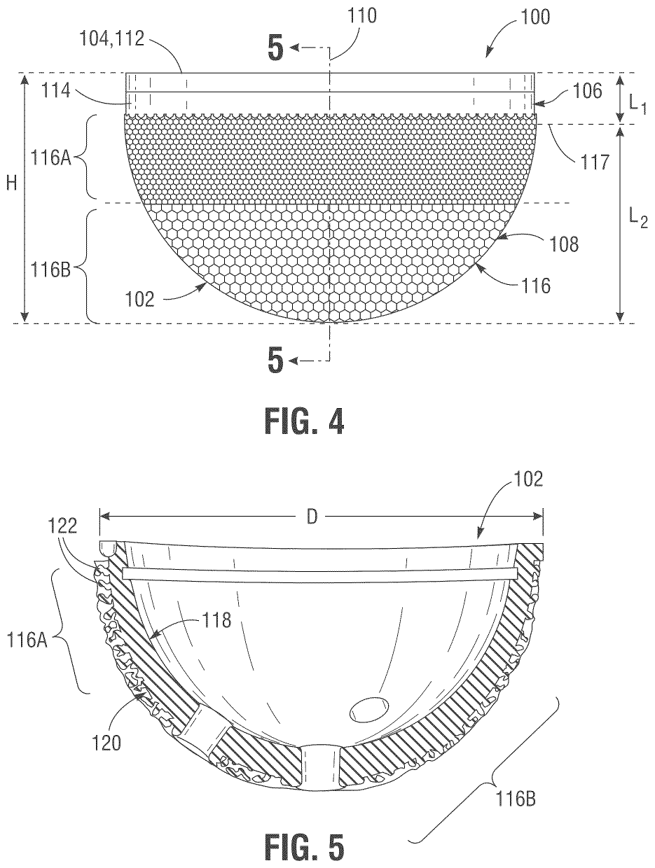

For example, FIG. 4 illustrates a representative embodiment of an acetabular cup 100 comprising a metallic main body 102 and a polymeric liner and/or insert member 104 positioned within the main body 102. When the main body 102 and the insert member 104 are assembled, the acetabular cup has a first, cylindrical end portion 106 and a second, hemispherical end portion 108. In an implantation orientation, the cylindrical first end portion 106 can be the distal end portion of the acetabular cup and the hemispherical second end portion 108 can be the proximal end portion. A longitudinal axis of the acetabular cup is indicated at 110. In the illustrated embodiment, the insert member 104 includes an annular or radial edge portion 112 that forms part of the cylindrical first end portion 106, and forms the distal end of the acetabular cup (in an implantation orientation). The main body 102 can comprise an annular portion 114 located distally of the radial edge portion 112 of the insert member 104 (downwardly along axis 110 in FIG. 4). The cylindrical first end portion 106 can have a length L₁ measured along the axis 110, and the hemispherical second end portion 108 can have a length (e.g., a radius) L₂ also measured along the axis 110. The overall height H of the acetabular cup 100 can be the sum of the length dimensions L₁ and L₂.

The annular portion 114 of the main body 102 can comprise a relatively smooth surface. The main body 102 can further comprise a porous bone-contacting structure generally indicated at 116, which can cover the entire or substantially the entire outer surface of the main body 102 distally of the annular portion 114. In certain embodiments, the porous structure 116 can cover the hemispherical second end portion 108, and can extend across the equator 117 of the second end portion and cover at least a portion of the cylindrical first end portion 106.

FIG. 5 illustrates a cross-sectional view of the main body 102. The main body 102 can comprise a solid shell or substrate 118. The porous structure 116 can comprise a three-dimensional structure formed on the radially outward surface 120 of the solid substrate 118. In particular embodiments, the three-dimensional structure can comprise a plurality of strut members 122 extending outwardly from the bulk material of the main body 102, such as outwardly from the outer surface 120 of the solid substrate 118.

In the illustrated embodiment, the porous structure 116 comprises two discrete, circumferentially extending surface regions referred to herein as zones or surface zones 116A and 116B. The first zone 116A is located proximally of the annular portion 114 (e.g., downwardly along the axis 110) and encompasses the equator 117 of the hemispherical second end portion 108. The second zone 116B is located proximally of the first zone 116A, and encompasses the pole or proximal end of the hemispherical second end portion 108.

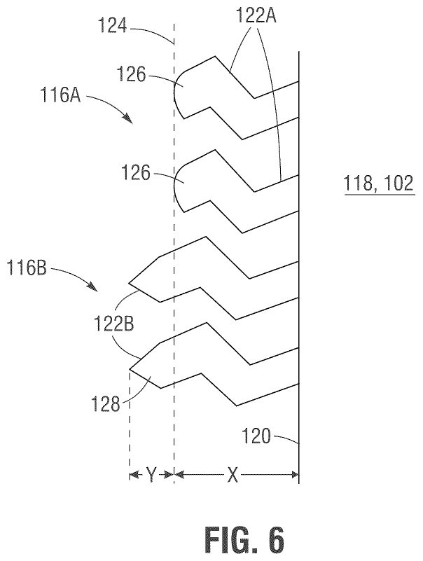

In certain embodiments, the surface roughness of the first zone 116A can be different from the surface roughness of the second zone 116B. For example, in certain embodiments the surface roughness of the second zone 116B can be greater than the surface roughness of the first zone 116A. In certain embodiments, the struts in the first zone 116A can have a different edge condition, end condition, and/or end structure than the struts in the second zone 116B. For example, FIG. 6 schematically illustrates the boundary between the first zone 116A and the second zone 116B, including a plurality of strut members 122A of the first zone 116A and a plurality of strut members 122B of the second zone 116B. In certain embodiments the strut members 122A in the first zone 116A can extend outwardly from the outer surface 120 of the substrate 118 by a specified distance X measured between the outer surface 120 of the substrate 118 and the dashed line 124. In certain embodiments, the specified distance X (e.g., the overall height of the strut members) can be 2% to 6% of the overall diameter D of the acetabular cup (FIG. 5), such as 2% to 5%, 3% to 5%, 3% to 6%, etc., of the diameter D. In certain embodiments, the height of the struts can be 0.5 mm to 3 mm, such as 0.5 mm to 2.5 mm, 0.5 mm to 2 mm, 1 mm to 2 mm, 1 mm to 3 mm, etc.

In certain embodiments, the end portions 126 of the strut members 122A can be flat, smooth, and/or rounded such that deviation from the specified distance 124, and thus the surface roughness in the first zone 116A, can be relatively low. The strut members 122B in the second zone 116B can be longer than the struts 122A, and can extend beyond the specified distance X. In certain embodiments, the strut members 122B can also comprise relatively angular, jagged, irregular, and/or pointed end portions 128. Accordingly, the surface roughness of the second zone 116B can be greater than the surface roughness of the first zone 116A. The pointed, irregular shapes of the end portions 128 of the strut members 122B can also contribute to an increased coefficient of friction with bone tissue as compared to the first zone 116A.

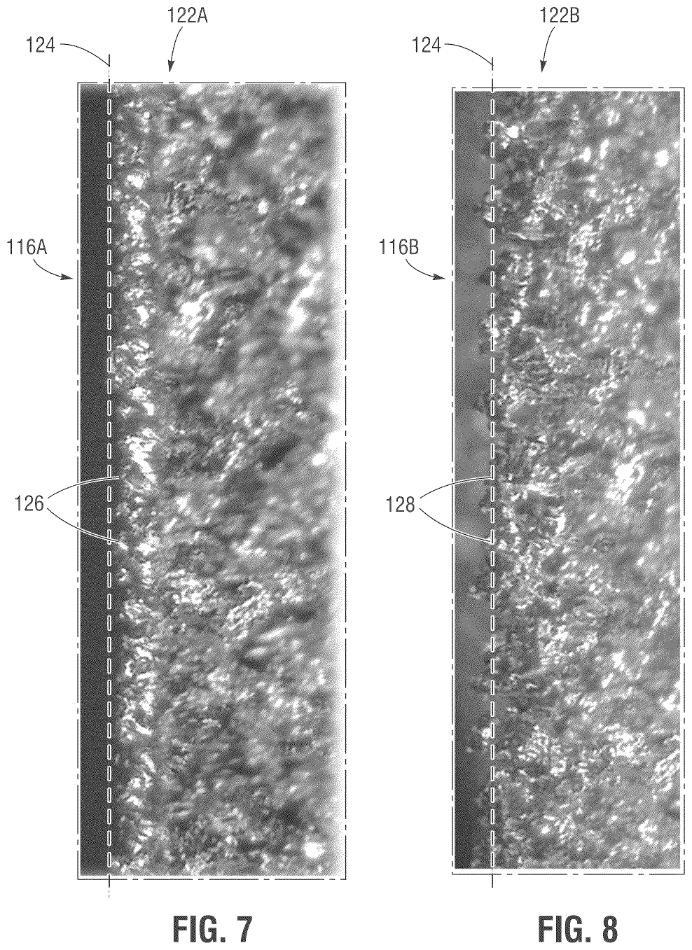

FIGS. 7 and 8 are micrographs illustrating end portions of strut members in the first zone 116A (FIG. 7) and in the second zone 116B (FIG. 8) of a representative example of the acetabular cup 100. As shown in FIG. 7, the struts 122A extend generally to the dashed line 124 and not beyond, and have relatively smooth, rounded end portions. Deviation of the strut members 122A from the dashed line 124 is generally to the right in FIG. 7 in a direction toward the solid substrate 118. Stated differently, the strut members 122A in the first zone 116A generally extend outwardly from the solid surface of the main implant body by the specified distance X or less. In certain examples, the radii of the end portions of the strut members 122A in the first zone 116A can be equal or substantially equal to the diameter of the struts (e.g., 0.1 mm to 10 mm).

Referring to FIG. 8, the struts 122B in the second zone 116B generally extend beyond the dashed line 124, and have angular, pointed end portions 128. Taking the dashed line 124 as a mean line, deviation of the strut members 122B is both to the left (away from the solid substrate 118) and to the right (toward the solid substrate 118), resulting in greater surface roughness and an increased coefficient of friction with bone. Referring again to FIG. 6, in certain examples at least a portion of the strut members 122B can extend beyond the specified distance X by a distance Y. In certain embodiments, the distance Y can be 0.5 mm or less, such as 0.3 mm or less, 0.1 mm or less, 0.1 mm to 0.5 mm, 0.1 mm to 0.3 mm, 0.3 mm to 0.5 mm, 10 μm to 500 μm, 10 μm to 400 μm, 10 μm to 300 μm, 10 μm to 200 μm, 10 μm to 100 μm, 50 μm to 100 μm, 50 μm to 200 μm, 50 μm to 300 μm, 50 μm to 400 μm, 50 μm to 500 μm, etc. In certain embodiments, the strut members 122B can extend beyond the specified distance X by varying amounts, resulting in increased surface roughness and a correspondingly higher coefficient of friction with bone.

The surface roughness of the porous structure 116 can be determined in a variety of ways. For example, in certain embodiments the arithmetic mean roughness parameter Ra of the porous structure 116 can be determined along a mean line, such as the dashed line 124 at the specified distance X from the solid substrate 118. The surface roughness can also be determined or expressed according to any of various other parameters, such as the range roughness parameter Rt, the root mean squared roughness parameter Rq, or any of various surface area roughness parameters.

In certain embodiments, the surface roughness of the first zone 116A can be such that the first zone 116A exhibits a coefficient of friction of 0.6 to 0.8 with cancellous bone tissue, such as 0.7 to 0.8, or 0.75 or less. In certain embodiments, the first zone 116A can exhibit a coefficient of friction of 0.6 to 0.8 with cancellous bone tissue, such as 0.7 to 0.8, or 0.75 or less. In certain embodiments, the surface roughness of the second zone 116B can be such that the second zone 116B exhibits a coefficient of friction of 0.8 to 1.0 with cancellous bone tissue, such as 0.85 to 0.95 or 0.8 or more. In certain embodiments, the second zone 116B can exhibit a coefficient of friction of 0.8 to 1.0 with cancellous bone tissue, such as 0.85 to 0.95 or 0.8 or more.

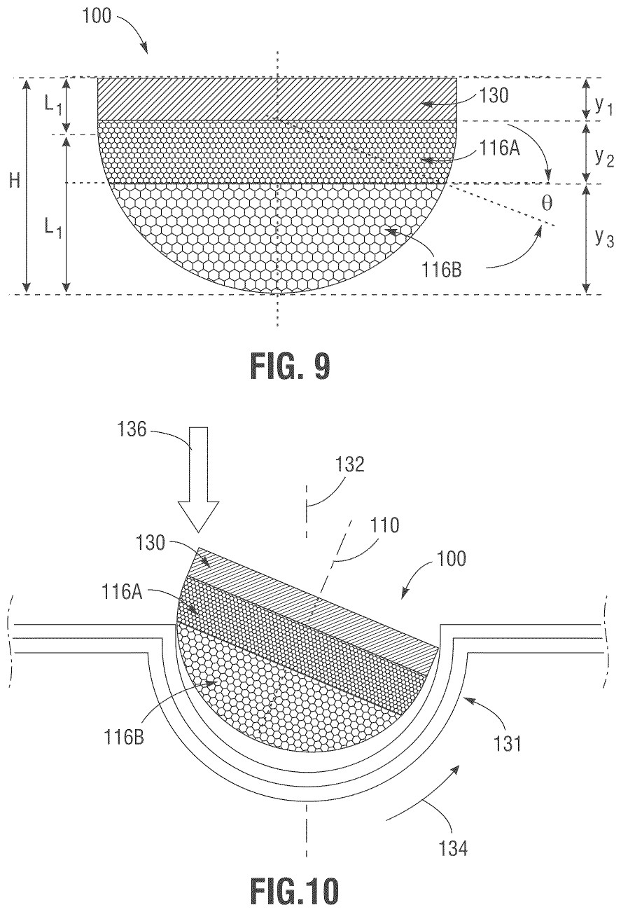

The extent of the first zone 116A and the second zone 116B as a proportion of the overall surface of the porous structure 116, and/or as a proportion of the overall height H of the implant, can vary. For example, FIG. 9 schematically illustrates the extent of the first zone 116A of the porous structure and the second zone 116B of the porous structure relative to the overall length H of the acetabular cup 100, according to one embodiment. The relatively low friction radial edge portion 112 of the insert member 104 and the annular portion 114 of the main body 102 are shown combined as a surface zone 130 at the first end portion 106. The height dimension of the surface zone 130 is denoted y₁, the height dimension of the first zone 116A of the porous structure is denoted y₂, and the height dimension of the second zone 116B of the porous structure is denoted y₃.

In certain embodiments, the height dimension y₁ of the surface zone 130 can be 5% to 30% of the overall height H, such as 10% to 30%, 20% to 30%, 10% to 25%, or 10% to 20%. In certain embodiments, the height dimension y₂ of the first zone 116A of the porous structure can be 10% to 50% of the overall height H, such as 10% to 40%, 20% to 50%, 20% to 40%, 10% to 30%, or 10% to 20%. In certain embodiments, the height dimension y₃ of the second zone 116B of the porous structure can be 30% to 70% of the overall height H, such as 30% to 60%, 40% to 70%, or 40% to 60%. In particular embodiments, the height dimension y₁ can be 20% of the overall height H, the height dimension y₂ can be 30% of the overall height H, and the height dimension y₃ can be 50% of the overall height H. In certain embodiments, the combined height dimensions y₁ and y₂ (e.g., collectively the “low friction” portion of the outer surface of the acetabular cup) can be 20% to 40% over the overall height H.

In use, varying the surface roughness of the implant body as described herein can allow rotation and/or reorientation of the implant about various axes after initial placement of the implant, and prior to impaction to its final position. In certain embodiments, by reducing the surface roughness of the portion(s) of the porous structure that contact native bone tissue first or early in the implantation process, the acetabular cup 100 can be rotated about nearly any axis. FIG. 9 illustrates rotation of the acetabular cup 100 by an angle θ relative to an axis extending out of the plane of the page. In certain embodiments, the angle θ can be 200 or more, depending upon the size of the reduced roughness zone of the porous surface.

For example, FIGS. 10-12 illustrate the ability to reposition the acetabular cup 100 provided by the varying surface roughness of the porous structure 116, and the corresponding variation in the coefficient of friction of the porous structure with bone. FIG. 10 illustrates initial placement of the acetabular cup 100 in a prepared native acetabulum 131, for example, after the cup has been lightly tapped into the prepared acetabulum and the impactor removed. In FIG. 10, the axis 110 of the acetabular cup 100 is misaligned with the axis 132 of the native acetabulum 131, and reorientation of the acetabular cup is indicated. As shown in FIG. 10, contact between the acetabular cup 100 and the native bone tissue of the acetabulum 131 is primarily between the surface zone 130 (e.g., the radial edge portion 112 and/or the annular portion 114) and the first zone 116A of the porous structure 116. In FIG. 10, only a relatively small portion of second zone 116B of the porous structure 116 is in contact with the native acetabulum 131 at the left side of the figure due to the longitudinal offset between the first zone 116A and the second zone 116B.

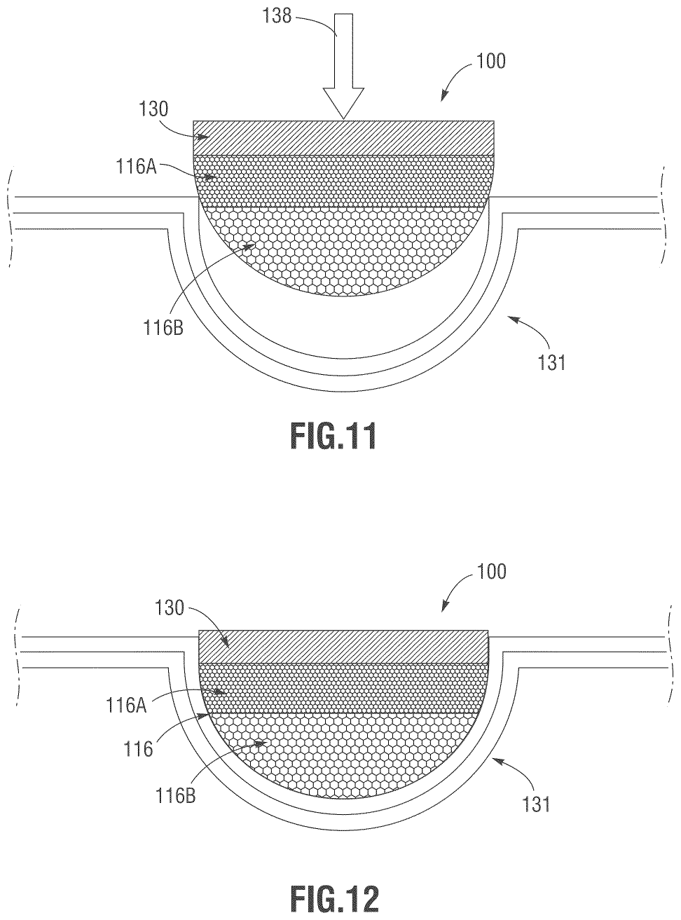

Accordingly, the axis 110 of the acetabular cup 100 can be aligned with the axis 132 of the native acetabulum 131 by rotating the acetabular cup in the direction of arrow 134. In certain embodiments, this can be accomplished by tapping the acetabular cup with an impactor at the location and in the direction indicated by arrow 136. The aligned acetabular cup 100 and native acetabulum 131 are illustrated in FIG. 11. Due to the relatively low surface roughness of the first zone 116A, the acetabular cup 100 can be rotated in the native acetabulum 131 to align their axes without damaging the preparation of the acetabulum, and without removing the acetabular cup from the acetabulum. Stated differently, the relatively low coefficient of friction of the first zone 116B of the porous structure can allow the acetabular cup 100 to be rotated in situ prior to impaction or press-fitting of the acetabular cup to its final position. Referring to FIG. 11, after alignment of the acetabular cup with the acetabulum, the impactor can be assembled back onto the acetabular cup 100 and the acetabular cup can be tapped or impacted into its final position in the direction of arrow 138. The final position of the acetabular cup 100 press-fitted into the native acetabulum 131 is shown in FIG. 12. In FIG. 12, the whole of the porous surface 116 including both the first zone 116A and the second zone 116B is shown in contact with the tissue of the native acetabulum 131. In the final implantation position, the zone 116A can be primarily in shear, and the zone 116B can be primarily in compression.

In certain embodiments, the solid shell 118 and the porous structure 116 can be made using three-dimensional printing or additive manufacturing techniques. For example, in certain embodiments the three-dimensional porous structure 116 can be formed on the solid shell 118 by applying metal powder or granules and sintering the powder together in a series of layers. In certain embodiments, the solid shell 118 can be molded or cast, and the porous structure 116 can be three-dimensionally printed on the surface of the solid shell 118. The variation in roughness between the various zones of the surface of the porous structure 116 can be provided by varying the shape and/or size of the end portions of the three-dimensionally printed struts in the various zones as described above.

In other embodiments, the porous structure 116 can have more than two zones, such as three zones, four zones, five zones, etc., each zone having a different surface roughness and/or exhibiting a different coefficient of friction with bone tissue. For example, the surface roughness of each zone can increase moving along the porous structure in the implantation direction. In yet other embodiments, the surface roughness can increase and decrease alternatingly along the surface of the porous structure in the implantation direction, depending upon the particular characteristics desired. The surface roughness of the porous structure 116 can also increase in the proximal direction from a first or minimum surface roughness at the distal edge of the porous structure 116 to a second or maximum surface roughness at the proximal edge (e.g., at the pole of the acetabular cup) in a continuous gradient.

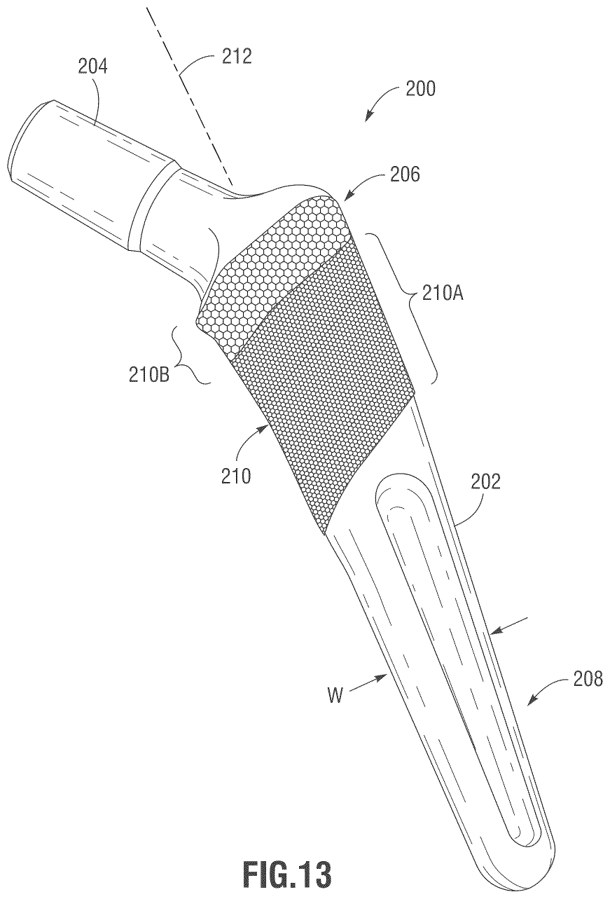

Porous structures with varying surface roughness can also be implemented on femoral stem prostheses. FIG. 13 illustrates an embodiment of a femoral stem prosthesis 200 including a stem member 202 and a neck member 204. The stem member 202 can comprise a proximal end portion 206 and a distal end portion 208. The neck member 204 can extend from the proximal end portion 206 of the stem member 202 at an angle to the longitudinal axis of the stem member. In some embodiments, the neck member 204 and the stem member 202 can be integrally formed from a single piece of material, or can be separately formed and fastened together depending upon the particular characteristics desired. In certain embodiments, the stem member 202 can have a generally tapered shape such that the distal end portion 208 has a smaller cross-sectional area than the proximal portion 206. Stated differently, a width, diameter, or thickness dimension W of the stem member 202 can increase in a direction from the distal end portion 208 to the proximal end portion 206.

The proximal end portion 206 can have a region comprising a circumferentially-extending porous structure 210 configured to promote bone ingrowth to create mechanical interlocking between the femur and the femoral stem prosthesis, as described above. In the illustrated embodiment, the porous structure 210 can comprise a plurality of regions or zones having different surface roughness and/or exhibiting different coefficients of friction with bone material. In certain examples, portion(s) of the porous structure 210 that contact native bone tissue early in the implantation process can comprise a relatively low surface roughness. Portion(s) of the porous structure 210 that contact bone tissue later in the implantation process or at final impaction of the implant can comprise a relatively higher surface roughness, and can be offset longitudinally from the zones of lower surface roughness. For example, in the illustrated embodiment the porous structure 210 comprises a circumferentially-extending first or distal zone 210A having a relatively lower surface roughness, and a circumferentially-extending second or proximal zone 210B having a relatively higher surface roughness. Thus, the surface roughness and/or the coefficient of friction between the porous structure 210 and bone can decrease moving along the porous structure in the direction of implantation (e.g., downwardly along axis 212).

In certain embodiments, the distal zone 210A can extend along 70% to 90% of the overall axial length of the porous structure 210 (e.g., as measured along the longitudinal axis 212 of the stem member 202). In certain embodiments, the distal zone 210A can extend along 75% to 85% or 80% of the overall axial length of the porous surface 210. In certain embodiments, the proximal zone 210B can extend along 10% to 30% of the overall axial length of the porous structure 210, such as 15% to 25%, 10% to 20%, or 20% of the overall axial length of the porous structure 210.

In certain embodiments, the surface roughness of the distal zone 210A can be such that the distal zone 210A exhibits a coefficient of friction with cancellous bone tissue of 0.6 to 0.8, such as 0.7 to 0.8 or 0.75 or less, similar to the zone 116A of the acetabular cup 100. In certain embodiments, the distal zone 210A can exhibit a coefficient of friction with cancellous bone tissue of 0.6 to 0.8, such as 0.7 to 0.8 or 0.75 or less. In certain embodiments, the surface roughness of the proximal zone 210B can be such that the proximal zone 210B exhibits a coefficient of friction of 0.8 to 1.0 with cancellous bone tissue, such as 0.85 to 0.95 or 0.8 or more. In certain embodiments, the proximal zone 210B can exhibit a coefficient of friction of 0.8 to 1.0 with cancellous bone tissue, such as 0.85 to 0.95 or 0.8 or more.

In certain embodiments, the native femur can be broached or reamed to create a canal that has a slightly smaller size (e.g., diameter) than the stem member 202. The stem member 202 can be inserted into the femoral canal to create an interference or frictional fit for establishing initial stability of the femoral stem prosthesis 200. As the stem member 202 is advanced into the femur, the relatively low roughness/friction distal zone 210A of the porous structure 210 can contact bone tissue of the femur before the relatively higher roughness/friction proximal zone 210B. In certain embodiments, at least the distal zone 210A, or both the proximal zone 210B and the distal zone 210A, can be primarily in shear.

In other embodiments, the porous structure 210 can comprise any number of zones having different roughness/frictional characteristics, as described above. The surface roughness of the porous structure 210 can also decrease in the distal direction from a first or maximum surface roughness at the proximal edge of the porous structure 210 to a second or minimum surface roughness at the distal edge in a continuous gradient.

In certain embodiments, the acetabular cup 100 and/or the femoral stem prosthesis 200 can be formed of any of various biocompatible metal materials. For example, in certain embodiments the prostheses can be formed of titanium alloys, such as ASTM F-136 (Ti-6Al-4V ELI Titanium Alloy). In other embodiments, the acetabular cup 100 and/or the femoral stem prosthesis 200 can be formed using other biocompatible metals such as cobalt chromium, stainless steel, and/or various composite materials or polymers.

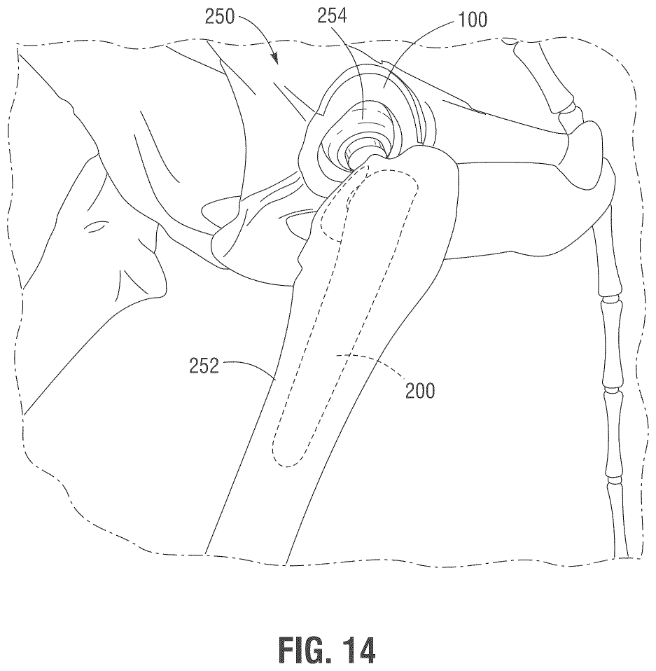

FIG. 14 illustrates an acetabular cup 100 configured according to the embodiment of FIG. 4 implanted in an acetabulum of a canine pelvis 250, and a femoral stem prosthesis 200 configured according to the embodiment of FIG. 13 implanted in the femur 252. A prosthetic femoral head 254 is shown coupled to the femoral stem prosthesis and received in the acetabular cup 100.

### Example 2: Canine Elbow Implant with Porous Structures Having Varying Coefficients of Friction with Bone

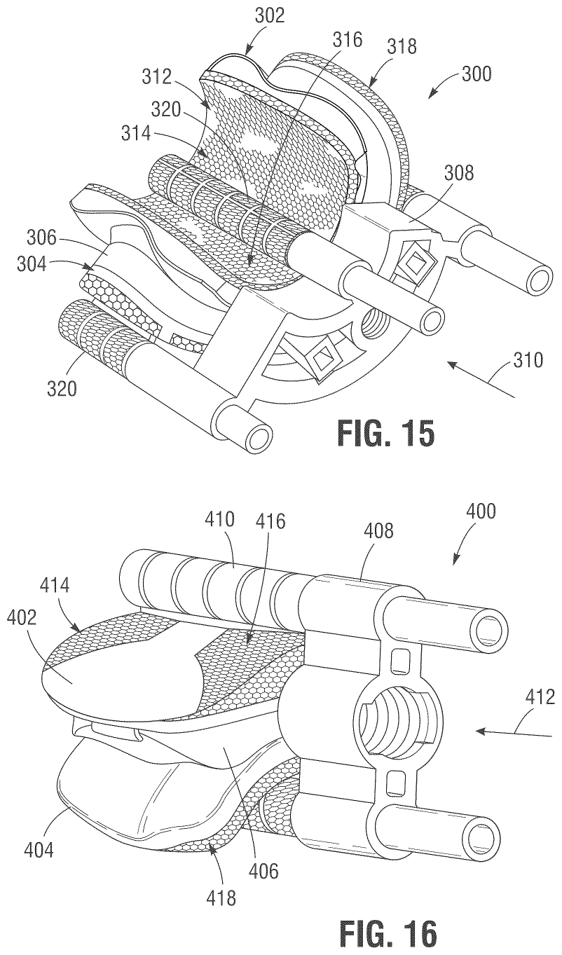

As noted above, porous structures with zones of differing roughness as described herein can be applicable to a variety of other orthopedic implants. For example, FIG. 15 illustrates an implant 300 configured to replace the canine elbow joint. The implant 300 can comprise a first, radially inner portion/member/assembly generally indicated at 302, and referred to hereinafter as a “first member.” The implant 300 can further comprise a second, radially outer portion/member/assembly generally indicated at 304 referred to hereinafter as a “second member,” and positioned around the first member 302. The first and second members 302, 304 can both be curved. A bearing member 306 is shown positioned between the first member 302 and the second member 304. A removable fixation guide member 308 is shown coupled to expandable post members 320 of the implant 300. The first member 302 can be configured for fixation (e.g., by bone screws) to the distal end portion of the humerus, and the second member 304 can be configured for fixation to the proximal end portions of the radius and the ulna. Arrow 310 indicates the direction of implantation or insertion of the implant 300 between the prepared humerus and the radius and ulna.

The first member 302 can comprise a porous structure 312 on the radially inner surface or aspect configured to contact the humerus when implanted. The porous structure 312 can comprise a three-dimensional porous structure according to any of the embodiments described herein. In certain embodiments, the porous structure 312 can have strut members with rounded end portions and relatively uniform height as described above with reference to FIGS. 6 and 7 such that the porous structure has a relatively low surface roughness and exhibits a relatively low coefficient of friction with bone tissue (e.g., 0.6 to 0.8, or 0.75 or less with cancellous bone tissue).

In certain embodiments, a medial portion or zone 314 of the porous structure 312 can comprise a relatively lower surface roughness and correspondingly lower coefficient of friction than a lateral portion or zone 316 of the porous structure. For example, in certain embodiments the lateral zone 316 of the porous structure 312 can comprise strut members with angular, pointed end portions and varying heights as described above with reference to FIGS. 6 and 8. In certain embodiments, the medial zone 314 of the porous structure 312 can contact or encounter bone before the lateral zone 316 during implantation of the implant 300, and the low surface roughness of the zone 316 can thereby facilitate implantation of the implant.

In certain embodiments, the radially outward aspect of the second member 304 can comprise a porous structure 318. In certain embodiments, the porous structure 318 can comprise a relatively low surface roughness and coefficient of friction with bone, or a medial zone and a lateral zone configured similarly to the medial and later zones described above with reference to the porous structure 312.

### Example 3: Canine Ankle Implant with Porous Structures Having Varying Coefficients of Friction with Bone

FIG. 16 illustrates another embodiment of an orthopedic implant 400 configured for replacement of the canine ankle joint. The implant 400 can comprise a curved first member 402 and a curved second member 404 with a bearing member 406 disposed between the members 402 and 404. The curvature of the first and second members 402, 404 can be such that the convex surfaces of the members are oriented in the dorsal direction away from the foot when implanted. A removable guide member 408 is shown coupled to expandable post members 410 of the first member 402 and the second member 404. Arrow 412 indicates the direction of implantation or insertion of the implant 400 between the prepared talus bone and the tibia and fibula.

The first member 402 can be configured for implantation at the distal end portion of the tibia and/or the fibula. The second member 404 can be configured for implantation at the proximal end portion of the talus bone. The radially outward (e.g., dorsal or proximal) aspect of the first member 402 can comprise one or a plurality of portions including three-dimensional porous structures according to any of the embodiments described herein. For example, in the illustrated embodiment the first member 402 comprises two porous structures, a porous structure 414 on a medial aspect or end portion of the first member 402, and a porous structure 416 on a lateral aspect or end portion of the first member 402. The porous structures 414 and 416 can be configured according to any of the porous structure embodiments described herein. In certain embodiments, the porous structures 414 and/or 416 can extend onto the medial or lateral surfaces, respectively, of the first member 402.

In certain embodiments, the medial porous structure 414 and the lateral porous structure 416 can both comprise strut members with rounded end portions and relatively uniform heights such that the porous structures 414 and 416 have a relatively low surface roughness and a relatively low coefficient of friction with bone (e.g., 0.6 to 0.8, or 0.75 or less with cancellous bone). In certain embodiments, the surface roughness and the coefficients of friction of the porous structures 414 and 416 can be different. For example, in certain embodiments the surface roughness and coefficient of friction of the medial porous structure 414, which can encounter bone first as the implant 400 is implanted, can be lower than the surface roughness and coefficient of friction of the lateral porous structure 416. In certain embodiments, the lateral porous structure 416 can comprise strut members with pointed end portions and varying heights, resulting in a relatively high surface roughness and a relatively high coefficient of friction with cancellous bone (e.g., 0.8 to 1.0, or 0.8 or more).

In certain embodiments, one or both of the porous structures 414 and/or 416 can comprise multiple regions or zones having different surface roughness and corresponding coefficients of friction with bone, as described above. In certain embodiments, the second member 404 can comprise a plurality of porous structures such as the porous structure 418 visible in FIG. 16, which can be configured similarly to the porous structures 414 and 416.

### Example 4: Canine Stifle Implant with Porous Structures Having Varying Coefficients of Friction with Bone

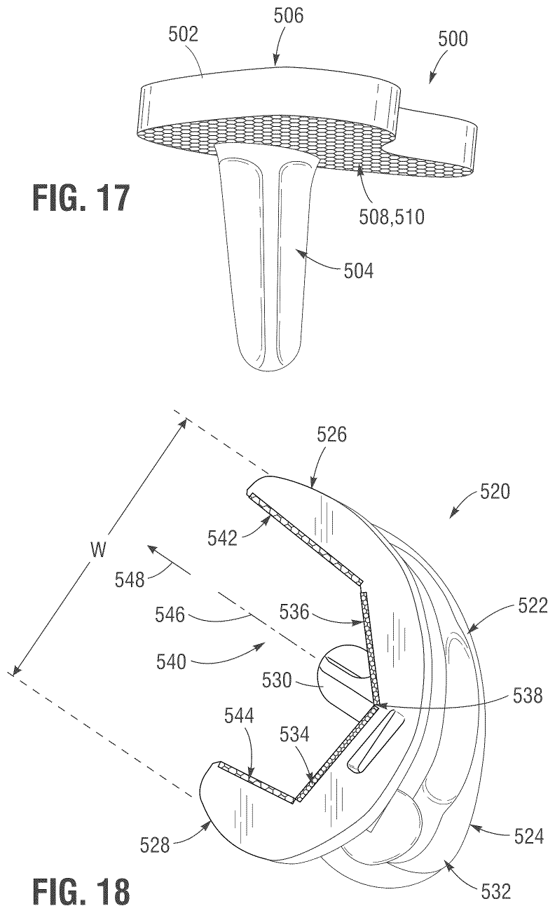

FIGS. 17 and 18 illustrate orthopedic implants configured to replace the canine stifle (knee) joint, according to one embodiment. FIG. 17 illustrates a tibial implant prosthesis 500 configured for implantation into the tibia to replace the tibial plateau and associated structures of the stifle joint. The tibial implant 500 can include a first platform member or portion 502 and a second stem member or portion 504 coupled to the platform member 502 and extending from the platform member 502 at an angle (e.g., 90°) to the primary surfaces of the platform member 502. The platform member 502 can comprise a first, proximal surface 506 and a second, distal surface 508 from which the stem member 504 extends. In certain embodiments, the distal surface 508 of the platform member 502 can comprise a porous structure 510, which can be configured according to any of the embodiments described herein. In certain embodiments, strut members of the porous structure 510 can comprise pointed end portions and varying heights such that the porous structure has a relatively high surface roughness, and a corresponding coefficient of friction of 0.8 to 1.0, or 0.8 or more, with cancellous bone tissue. In the final implantation position, the surface 08 and the porous structure 510 can be primarily in compression.

When the stem member 504 is inserted into the prepared tibia, the porous structure 510 can contact the proximal end portion of the tibia, and the relatively high surface roughness of the porous structure 510 can frictionally engage the tibia to inhibit motion of the implant 500. In certain embodiments, the porous structure 510 can comprise zones of greater or reduced roughness, as described above.

FIG. 18 illustrates a femoral implant prosthesis 520 configured to be coupled to the distal end portion of the femur and engage the tibial implant prosthesis 500 to form a replacement stifle joint. The femoral prosthesis 520 can comprise a curved or U-shaped main body 522 having a first or base portion 524, a second or cranial portion 526, and a third or caudal portion 528. The base portion 524 can be a first end portion of the implant and the cranial and caudal portions 526 and 528 can be a second end portion of the implant. The cranial portion 526 and the caudal portion 528 can extend from opposite sides of the base portion 524 (e.g., proximally in the implantation orientation), and can be spaced apart so as to define a gap 540 configured to receive the distal end portion of the femur. A fixation member 530 can extend from the base portion 524 between the cranial portion 526 and the caudal portion 528, and in the same general direction as the cranial and caudal portions (e.g., proximally in the implantation orientation). The fixation member 530 can be received in the femoral canal when the femoral prosthesis 520 is implanted.

The femoral implant prosthesis 520 can define a curved outer surface generally indicated at 532. The outer surface 532 can be continuous along the cranial portion 526, the base portion 524, and the caudal portion 528, and can be configured to engage the tibial prosthesis 500 of FIG. 17 (or a bearing therebetween) and permit sliding motion of the tibial prosthesis 500 relative to the femoral prosthesis 520 during motion of the tibia.

The inner aspect of the U-shaped main body 522 can comprise a plurality of bone-contacting surfaces or facets. For example, the base portion 524 can comprise two inner surfaces 534 and 536 angled toward each other and meeting at an apex 538. The cranial portion 526 can comprise an inner surface 542, and the caudal portion 528 can comprise an inner surface 544 facing the inner surface 542 of the cranial portion 526 across the gap 540. In certain embodiments, each of the inner surfaces 534, 536, 542, and/or 544 can comprise three-dimensional porous structures configured according to any of the embodiments described herein. For example, in certain embodiments the porous structures of the surfaces 534 and 536 of the base portion 524 can comprise strut members with pointed end portions and varying heights such that the porous structures have a relatively high surface roughness, and a corresponding relatively high coefficient of friction with bone (e.g., 0.8 to 1.0, or 0.8 or more, with cancellous bone). In certain embodiments, the porous structures of the surfaces 542 and 544 of the cranial portion 526 and the caudal portion 528, respectively, can comprise strut members with rounded end portions and relatively uniform heights such that the porous structures have a relatively low surface roughness and a corresponding relatively low coefficient of friction with bone (e.g., 0.6 to 0.8, or 0.75 or less, with cancellous bone). In certain embodiments, the porous structures of the surfaces 534, 536, 542, and 544 can be a single, continuous porous structure.

In certain embodiments, a width or thickness dimension W of the main body 522 measured in the cranial-caudal direction (e.g., perpendicular to the longitudinal axis 546) can increase from the base portion 524 moving along the longitudinal axis 546 of the implant in the direction indicated by arrow 548, which can be the implantation direction. Accordingly, the surface roughness/coefficient of friction of the porous structures can decrease moving in the implantation direction as the thickness dimension W increases. As the femoral implant prosthesis is advanced over the distal end portion of the femur, the porous structures at the surfaces 542 and 544 can contact bone tissue initially, followed by the surfaces 534 and 536 at final impaction/placement of the implant. The surfaces 542 and 544 can slide along native bone tissue of the femur during placement, aided by the relatively low surface roughness/friction of the porous structures. Once at its final position, the higher roughness/friction porous structures at the surfaces 534 and 536 can contact the bone and aid in retaining the implant in place. As in the previously described embodiments, the three-dimensional porous structures can provide for bone ingrowth at each of the surfaces 534, 536, 542 and 544 independent of the surface roughness of the porous structures to facilitate long term stability of the implant. At its final position, the surfaces 542 and 544 can be primarily in shear, and the surfaces 534 and 536 can be primarily in compression.

In certain embodiments, the porous structures of any or all of the surfaces 534, 536, 542 and/or 544 can comprise zones of varying surface roughness/friction, as described above. For example, in certain embodiments one or more of the surfaces 534, 536, 542 and/or 544 can comprise a lower roughness zone at a dorsal or proximal end of the surface (e.g., along the axis 546) where initial contact with bone is made during implantation, and a higher roughness zone at a ventral or distal end of the surface. In certain embodiments, the surface roughness of the porous structures can increase along the surfaces 534, 536, 542 and/or 544 in a distal direction in a continuous gradient.

### Example 5: Experimental Determination of Coefficient of Friction of Porous Structures

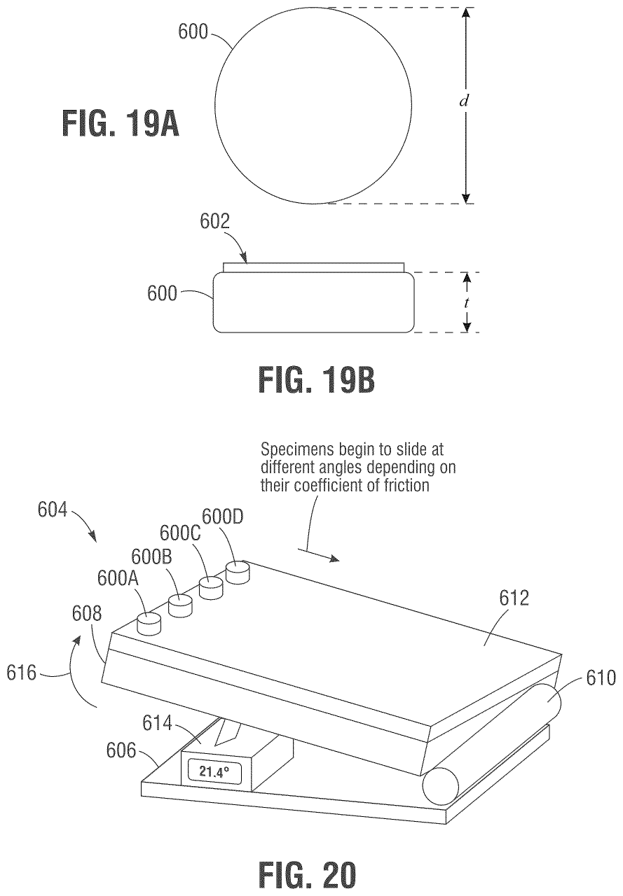

The following example describes a representative test apparatus and method by which the coefficient of friction of a porous structure, such as any of the porous structures described herein, can be determined with respect to bone tissue. FIGS. 19A and 19B illustrate a representative test member or coupon 600 configured as a circular disk. In a particular embodiment, the disk 600 had a diameter d of 28 mm and a thickness t of 10 mm, and were made of titanium 6Al-4V alloy. A test specimen 602 can be formed as a material layer on one surface of the test member 600. The test specimen 602 can be, for example, a three-dimensional porous structure according to any of the embodiments described herein.

FIG. 20 illustrates a test apparatus 604 including a base 606 and a platform member 608 rotatably coupled to the base 606 at one end by a coupling configured as a hinge 610. The platform 608 comprises a surface layer 612. The surface layer 612 can comprise any material with which the coefficient of friction is to be determined. In a particular embodiment, the surface layer 612 was made of simulated cancellous bone comprising polyurethane foam. The angle of the platform 608 relative to the base 606 can be determined by an inclinometer or tilt sensor, and displayed on a digital display 614.

The coefficient of static friction between the material of the test specimen 602 and the material of the surface layer 612 of the platform 608 can be determined by placing the disk 600 on the platform 608 with the test specimen 602 in contact with the surface layer 612. The platform 608 can then be inclined relative to the base 606 by rotating the free end of the platform upwardly in the direction indicated by arrow 616, and noting the angle of the platform at which the disk 600 begins to slide along the surface layer 612.

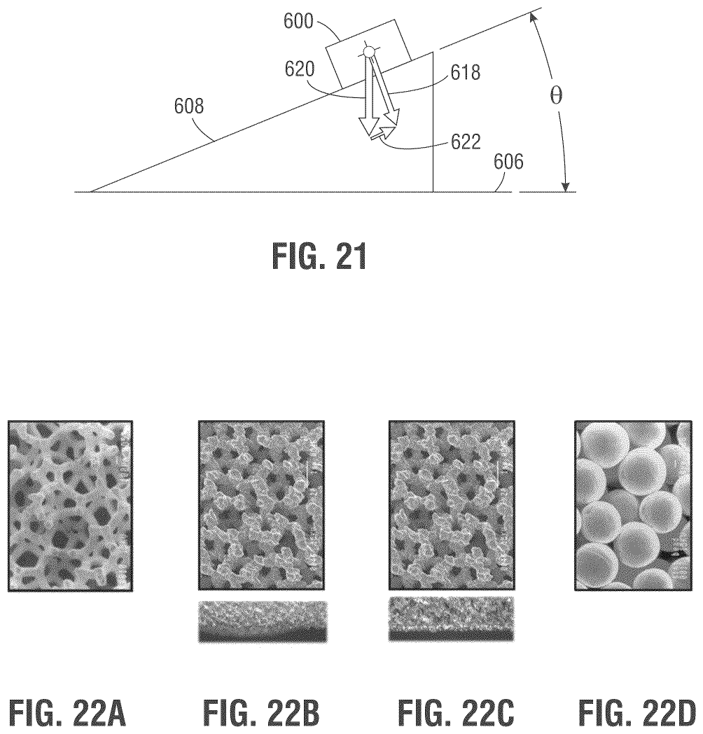

FIG. 21 illustrates a diagram of the forces acting on a disk 600 resting on the inclined platform 608. Arrow 618 represents the normal force F_{N} between the disk 600 and the platform 608. Arrow 620 represents the gravitational force F_{g} of the disk (mass×gravity), and arrow 622 represents the friction force F_{f}. The angle θ is the angle of the platform 608 relative to the base 606.

The normal force F_{N} can be determined by the following equation: F_{N}=mg sin θ, where m is the mass of the disk 600 and g is the acceleration due to gravity.

The friction force F_{f} can be determined by the following equation: F_{f}=mg cos θ.

The coefficient of static friction can be determined by the following equation:

$$μ = \frac{{F}_{f}}{{F}_{N}} .$$

Returning to FIG. 20, in one example four test members 600A-600D were fabricated. Each test member had the same surface area, the same mass, and different test specimens formed on surfaces of the test members. The test specimen on the test member 600A was a Trabecular Metal® porous tantalum structure available from Zimmer Biomet. A micrograph of the porous tantalum structure is shown in FIG. 22A. A three-dimensionally printed porous structure with titanium strut members including irregular, pointed end portions and varying heights as described with reference to zone 116B of FIGS. 4-6 was formed as the test specimen on test member 600B. A micrograph of the three-dimensional porous structure of test member 600B is shown in FIG. 22B. A three-dimensionally printed porous structure with titanium strut members including relatively uniform height, rounded end portions as described with reference to zone 116A of FIGS. 4-6 was formed as the test specimen on test member 600C. A micrograph of the three-dimensional porous structure of test specimen 600C is shown in FIG. 22C. A layer of sintered titanium beads having a mean diameter of approximately 550 m was formed as the test specimen on test member 600D. A micrograph of the sintered titanium beads of test specimen 600D is shown in FIG. 22D.

In a test, the test member 600A began to slide along the surface layer 612 at an angle of approximately 54°. The coefficient of static friction between the porous tantalum structure of the test member 600A and the polyurethane foam imitation cancellous bone material of the surface layer 612 was determined to be approximately 0.9.

In a test, the test member 600B began to slide along the surface layer 612 at an angle of approximately 51°. The coefficient of static friction between the relatively high surface roughness three-dimensional porous structure of test member 600B and the polyurethane foam imitation cancellous bone material of the surface layer 612 was determined to be approximately 0.87.

In a test, the test member 600C began to slide along the surface layer 612 at an angle of approximately 43°. The coefficient of static friction between the relatively low surface roughness three-dimensional porous structure of test member 600C and the polyurethane foam imitation cancellous bone material of the surface layer 612 was determined to be approximately 0.72.

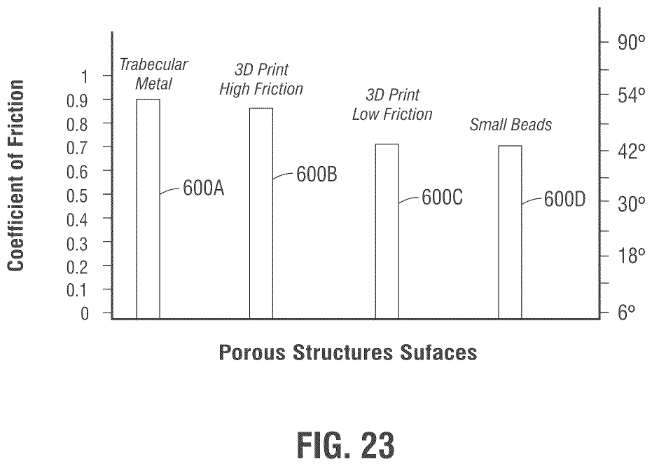

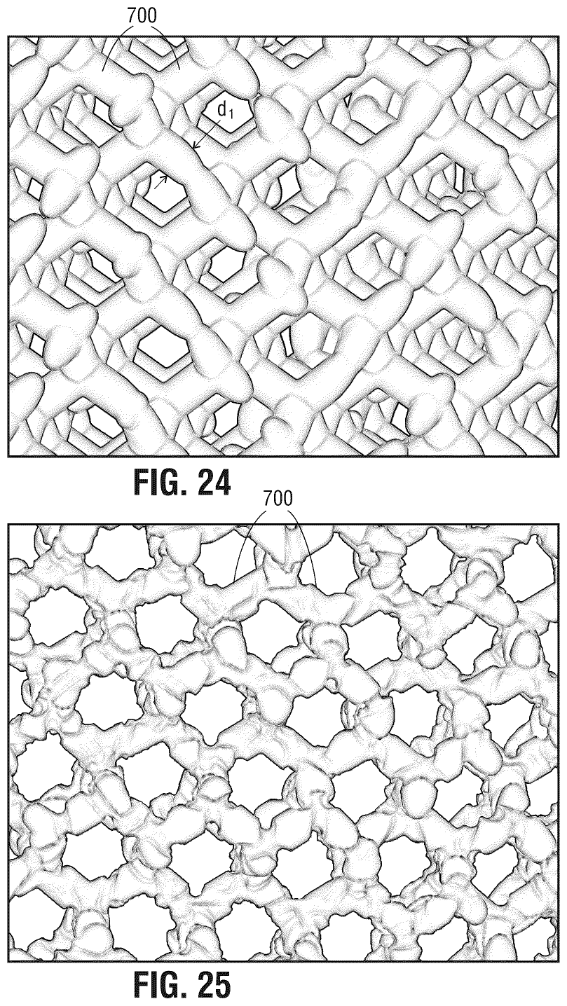

In a test, the test member 600D began to slide along the surface layer 612 at an angle of approximately 41°. The coefficient of static friction between the sintered titanium beads of the test member 600D and the polyurethane foam imitation cancellous bone material of the surface layer 612 was determined to be approximately 0.7. The results of each test are illustrated graphically in the chart of FIG. 23.

The three-dimensional porous structure embodiments and their placement at various locations on orthopedic implants as described herein can provide a number of significant advantages over existing orthopedic implants. For example, varying length of the strut members and the shape of their end portions to vary the surface roughness of the porous structure allows the coefficient of friction of the porous structure with bone to be controlled. Thus, one or more portions of a porous structure located on an implant surface which contacts native bone tissue early in the implantation process can have a relatively low surface roughness (e.g., struts of the porous structure can have rounded end portions and a generally uniform height), and a correspondingly low coefficient of friction with bone. This can facilitate not only insertion of the implant into the bone, but also repositioning of the implant mid-implantation because the relatively low coefficient of friction between the porous structure and the bone tissue reduces damage to the preparation as the implant is adjusted.

Additionally, one or more portions of a porous structure located on implant surfaces that come into contact with bone at or near the final position of the implant can have a relatively high surface roughness (e.g., struts of the porous structure can have angled and/or pointed end portions, and varying heights), and a correspondingly higher coefficient of friction with bone. Thus, when driven or impacted to its final position, these higher surface roughness zones of the porous structure can engage native bone tissue with a relatively high coefficient of friction to retain the implant at its final position. Because both the low surface roughness and the high surface roughness portions of the porous structure can have the same or similar cellular configuration/geometry (e.g., the same or similar strut diameter, pore size, etc.), bone can grow into all zones of the porous structure regardless of the surface roughness of the various zones to promote long term fixation of the implant. The shape of the struts of the porous structure, and thus the surface roughness and coefficient of friction, can be precisely controlled using additive manufacturing techniques when the porous structure is formed.

### Example 6: Orthopedic Implants with Porous Lattice Structures

FIGS. 24-26 illustrate various examples of an additively manufactured porous structures that can be used in combination with any of the orthopedic implants described herein. The particular porous structures shown are lattices comprises a plurality of struts 700. The struts are arranged in a diamond cubic structure in which planar meshes and/or two-dimensional sheets of hexagonal unit cells are interconnected to form a three-dimensional lattice structure. In other examples, the unit cells can have any shape (e.g., rectangular, triangular, octagonal, etc.).

FIG. 24 illustrates an example of a porous structure including struts 700 that are shaped and sized to exhibit relatively low friction with bone. The struts 700 have a specified diameter d₁ along their lengths. The diameter of the struts 700 in FIG. 24 can be relatively uniform throughout the lattice, and the struts can have relatively smooth outer surfaces. In certain examples, end portions of the struts can be rounded to a radius that is equal or substantially equal to the radius of the struts. This produces relatively smooth, round end portions on the struts and can avoid flat portions and sharp edges.

In certain examples, a porous structure such as shown in FIG. 24 comprising a titanium alloy (e.g., Ti-6Al-4V) and with a strut diameter d₁ of 0.3 mm can have a coefficient of friction μ with cancellous bone of 0.3 to 0.6, such as 0.35 to 0.55, 0.4 to 0.6, 0.4 to 0.5, etc. In a particular example, the coefficient of friction μ can be 0.45 with cancellous bone. The porous structure in FIG. 24 can thus lend itself to portions of an implant surface where low to medium friction with bone is desired.

FIG. 25 illustrates another example of a porous structure in which the struts 700 are shaped and sized for relatively high friction with bone. For example, the struts 700 can a have plurality of prominences, projections, and/or bosses interspersed with recesses that provide the struts with a rough, irregular, textured surface. The prominences and recesses can be formed in a pattern or can be randomly distributed. The prominences and recesses can have a uniform size and/or shape, or can have different shapes. The prominences can be angular and/or sharp, or can be rounded. In the example of FIG. 25, the prominences and recesses on the struts 700 are formed using a Voronoi debossing technique.

In certain examples, a porous structure such as shown in FIG. 25 comprising a titanium alloy (e.g., Ti-6Al-4V) and with a strut diameter d₁ of 0.3 mm can have a coefficient of friction μ with cancellous bone of 0.6 to 1.1, such as 0.7 to 1.1, 0.8 to 1.1, 0.7 to 1, etc. In a particular example, the coefficient of friction μ can be 0.97 with cancellous bone. The porous structure in FIG. 25 can thus lend itself to portions of an implant surface where medium to high friction with bone is desired.

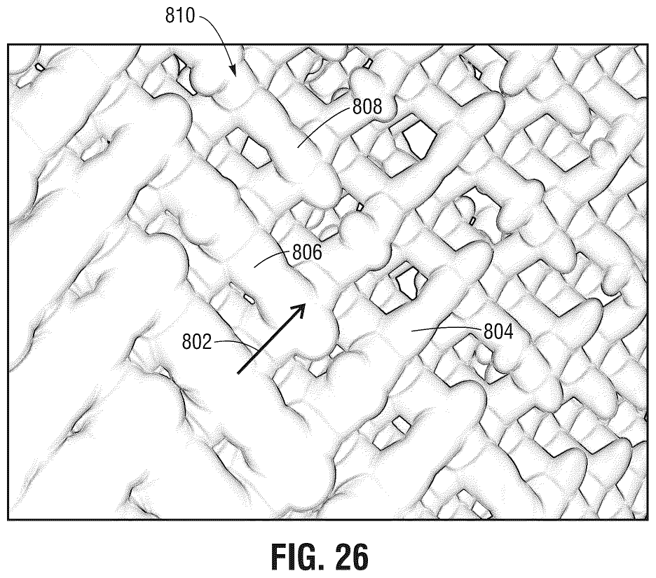

In certain examples, various porous lattice structures can be formed in different regions on the surface of an implant. The porous structures can also transition from a relatively low friction configuration to a relatively high friction configuration over a selected distance, and in one or a plurality of directions (e.g., in the direction of implantation). The surface of an implant can also transition from a solid bulk material to a porous structure over a selected distance, and in one or a plurality of directions. For example, FIG. 26 illustrates a portion of an implant in which a porous structure gradually develops from monolithic bulk material moving in a direction from the lower left corner toward the upper right corner of the figure along arrow 802. The arrow 802 represents the selected direction of the transition from a solid material to a porous structure. Zones in which such transitions occur are referred to hereinafter as transition zones, generally indicated at 810 in FIG. 26. The porous structure on the surface of the implant can be said to “fade in” from a solid surface to a porous structure in the transition zone 810.

This transition can be accomplished by varying the diameter of the struts in a gradient that extends in a particular direction or directions. For struts with longitudinal axes oriented in the selected direction, the diameter of the struts can vary along their axes. For struts oriented in other directions, the diameter of the struts can change by a selected amount in increments moving in the selected direction. For example, with reference to FIG. 26, struts such as a representative strut 804 having an axis oriented parallel to the arrow 802 can have a diameter that decreases moving along the strut in the direction of the arrow 802. Struts oriented in other directions, such as representative struts 806 and 808 located in the same plane and oriented at a 90° angle to the arrow 802 can have diameters that decrease by a specified increment with each sequential rung of struts in the direction of arrow 802.

For example, the struts oriented parallel to the direction of the transition such as strut 804 can vary from a relatively thick first diameter at one end of the transition zone 810 to a thinner second diameter. In certain examples, the second diameter can be a specified diameter of the struts in the fully developed porous lattice, where, for example, a specified gap and/or pore size is achieved in the porous structure. In at least a portion of the transition zone 810, the diameter of the struts can be sufficient to close the pores between the struts while still exhibiting strut-like structure on the surface. Stated differently, the initial diameter of the struts can be sufficiently large such that the struts at least partially merge together. As the diameter of the struts decreases in the selected direction (e.g., along arrow 802), gaps and/or open volumes can appear between the struts and gradually grow in size until the lattice structure is fully developed with a specified strut diameter and pore size.

In certain examples, a pore size of the lattice can be 0.1 mm to 2 mm, such as 0.1 mm to 1.5 mm, 0.5 mm to 2 mm, 0.5 mm to 1.5 mm, 0.1 mm to 1 mm, etc. In a particular example, the struts oriented parallel to the direction of the transition along arrow 802 can vary from a first diameter of 0.6 mm to a second diameter of 0.3 mm over a distance of 1 mm to 50 mm, such as 1 mm to 40 mm, 1 mm to 30 mm, 1 mm to 20 mm, 1 mm to 10 mm, 1 mm to 5 mm, 10 mm to 50 mm, 10 mm to 40 mm, 10 mm to 30 mm, 10 mm to 20 mm, etc., along the arrow 802. For struts in other orientations such as 806 and 808, the strut diameter can decrease from a first diameter of 0.6 mm to a second diameter of 0.3 mm in increments of 0.1 mm to 20 mm, such as 1 mm to 20 mm, 1 mm to 10 mm, 1 mm to 5 mm, 1 mm to 3 mm, 1 mm to 2 mm, 0.5 mm to 20 mm, 0.5 mm to 10 mm, 0.5 mm to 5 mm, 0.5 mm to 3 mm, 0.5 mm to 2 mm, 0.5 mm to 1 mm etc., over sequential rungs of struts in the direction of arrow 802.

In certain examples, transition zones such as the transition zone 810 can exhibit a coefficient of friction with bone of 0.2 to 0.6, such as 0.2 to 0.5, 0.2 to 0.4, 0.3 to 0.6, 0.3 to 0.5, etc.

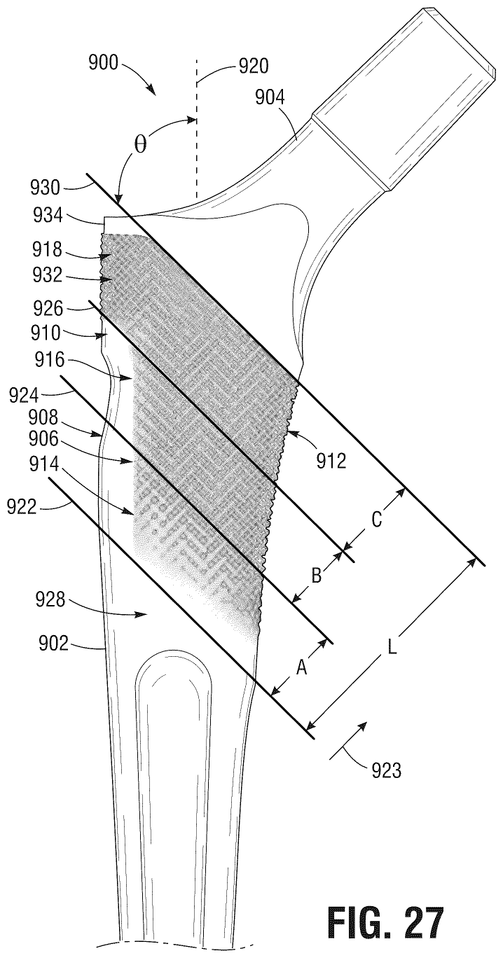

The porous structures above can be incorporated into any of the orthopedic implants described herein. For example, FIG. 27 illustrates another example of a femoral stem prosthesis 900 including a stem member 902, a neck member 904, and a porous lattice structure 906 formed primarily on a proximal end portion 908 of the stem member. The stem member 902 can comprise a lateral aspect and/or surface 910 and a medial aspect and/or surface 912.

The porous lattice structure 906 can comprise a plurality of zones configured to exhibit different coefficients of friction with bone. The zones can incorporate various of the porous lattice structures described above with reference to FIGS. 24-26, and/or any of the other porous structures described herein. In the illustrated example, the porous structure 906 can have a first, relatively low friction zone 914 (also referred to as a first zone and a first circumferentially-extending zone), a second, moderate friction zone 916 (also referred to as a second zone and a second circumferentially-extending zone), and a third, relatively high friction zone 918 (also referred to as a third zone and a third circumferentially-extending zone). In the illustrated example, the zones are oriented at an angle θ to the longitudinal axis 920 of the stem 902. In certain examples, the angle θ can be 20° to 90°, such as 30° to 60°, or 450 as in FIG. 27.

In the illustrated example, the low friction zone 914 can be a transition zone similar to FIG. 26 in which the porous structure 906 gradually develops from the bulk material of the stem 902. Line 922 indicates the cross-sectional plane at which the transition zone begins, referred to herein as the initiation plane. Arrow 923 indicates the direction of the transition. In FIG. 26, the arrow 923 is perpendicular or substantially perpendicular to the initiation plane 922, and at an angle of 45° to the axis 920 of the stem 902, although other configurations are possible. The coefficient of friction of the low friction zone 914 with cancellous bone can be as given above with reference to FIG. 26.

Line 924 indicates the cross-sectional plane at which the porous lattice structure 906 reaches a set of specified parameters for strut diameter and pore size. The plane 924 indicates the beginning of the moderate friction zone 916. In certain examples, the porous structure in the moderate friction zone 916 can be configured similarly to the structure described with reference to FIG. 24, and can have a similar coefficient of friction with cancellous bone.

Line 926 indicates the cross-sectional plane at which the porous structure 906 transitions to a high friction configuration. The plane 926 can thus mark the beginning of the high friction zone 918. In certain examples, the porous structure in the high friction zone 918 can be configured similarly to the structure described with reference to FIG. 25, and can have the coefficients of friction described with reference to FIG. 25.

The various zones 914-918 of the porous structure can extend at least partially around the perimeter and/or circumference of the implant body. In the example illustrated in FIG. 27, the low friction zone 914 and the moderate friction zone 916 can both extend circumferentially from the edge of the lateral surface 910 across the cranial surface 928, around the medial aspect or surface 912, and cross the caudal surface to the opposite edge of the lateral surface 910. The high friction zone 918 can extend circumferentially around the proximal end portion of the stem such that the high friction zone is continuous around full extent of the implant. Additionally, the circumferential edges of the high friction zone 918 can be defined at least in part by the plane 926 on the distal aspect and by a plane 930 along the proximal aspect. The plane 930 can mark the transition from the porous structure 906 back to a solid bulk material, and the beginning of the distal portion of the neck 904. A lateral portion 932 of the high friction zone 918 can extend around the lateral aspect 910 of the stem, and can be bounded on the proximal edge by a circumferentially-extending flange 934 of solid material. The high friction zone 918 extending around the full extent of the stem 902 can provide 360° engagement with bone in cases where this may be advantageous in view of the patient's anatomy. In certain examples the solid material surface on the lateral aspect 910 in the regions of the low friction zone 914 and the moderate friction zone 916 can provide improved fatigue life and resistance to crack propagation, such as in examples where the implant is formed by additive manufacturing techniques.

The porous structure 906 can extend along a length L defined in a direction parallel to the arrow 923 (e.g., in the direction of the decrease in the strut diameter). The low friction zone 914 can thus extend a distance A in the direction of arrow 923. The moderate friction zone 916 can extend a distance B in the direction of arrow 923. The high friction zone 918 can extend a distance C in the direction of arrow 923. In certain examples, A+B+C=L.

In certain examples, the length A of the low friction zone 914 can be 5% to 50% of the overall length L of the porous structure 906, 5% to 40% of the overall length L of the porous structure 906, 5% to 30% of the overall length L of the porous structure 906, 5% to 20% of the overall length L of the porous structure 906, 5% to 10% of the overall length L of the porous structure 906, 10% to 50% of the overall length L of the porous structure 906, 10% to 40% of the overall length L of the porous structure 906, 10% to 30% of the overall length L of the porous structure 906, 10% to 20% of the overall length L of the porous structure 906, 20% to 50% of the overall length L of the porous structure 906, 20% to 40% of the overall length L of the porous structure 906, 20% to 30% of the overall length L of the porous structure 906, 30% to 50% of the overall length L of the porous structure 906, 30% to 40% of the overall length L of the porous structure 906, etc. In one particular example, the length A of the low friction zone 914 can be 33% of the overall length L of the porous structure 906.

In certain examples, the length B of the moderate friction zone 916 can be 5% to 50% of the overall length L of the porous structure 906, 5% to 40% of the overall length L of the porous structure 906, 5% to 30% of the overall length L of the porous structure 906, 5% to 20% of the overall length L of the porous structure 906, 5% to 10% of the overall length L of the porous structure 906, 10% to 50% of the overall length L of the porous structure 906, 10% to 40% of the overall length L of the porous structure 906, 10% to 30% of the overall length L of the porous structure 906, 10% to 20% of the overall length L of the porous structure 906, 20% to 50% of the overall length L of the porous structure 906, 20% to 40% of the overall length L of the porous structure 906, 20% to 30% of the overall length L of the porous structure 906, 30% to 50% of the overall length L of the porous structure 906, 30% to 40% of the overall length L of the porous structure 906, etc. In one particular example, the length B of the moderate friction zone 916 can be 33% of the overall length L of the porous structure 906.

In certain examples, the length C of the high friction zone 918 can be 5% to 50% of the overall length L of the porous structure 906, 5% to 40% of the overall length L of the porous structure 906, 5% to 30% of the overall length L of the porous structure 906, 5% to 20% of the overall length L of the porous structure 906, 5% to 10% of the overall length L of the porous structure 906, 10% to 50% of the overall length L of the porous structure 906, 10% to 40% of the overall length L of the porous structure 906, 10% to 30% of the overall length L of the porous structure 906, 10% to 20% of the overall length L of the porous structure 906, 20% to 50% of the overall length L of the porous structure 906, 20% to 40% of the overall length L of the porous structure 906, 20% to 30% of the overall length L of the porous structure 906, 30% to 50% of the overall length L of the porous structure 906, 30% to 40% of the overall length L of the porous structure 906, etc. In one particular example, the length C of the high friction zone 918 can be 33% of the overall length L of the porous structure 906.

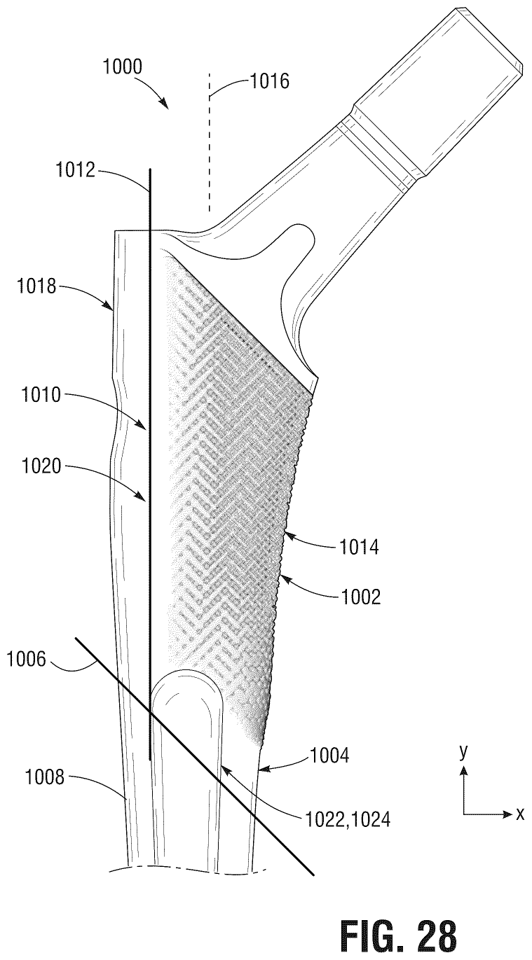

The coefficient of friction of the porous structure can thus decrease moving along the stem of the implant in the direction of implantation (e.g., downwardly along the longitudinal axis in FIG. 27). This decrease in frictional coefficient can hold true at any point along the entire circumference of the implant, as in FIG. 27, or along a portion thereof, such as 60% to 90% of the circumference or more, as in FIG. 28.

In certain examples, the porous structure can comprise multiple transition zones in which the porous lattice gradually develops along multiple axes and/or originates from multiple planes in different orientations. For example, FIG. 28 illustrates another example of a femoral stem prosthesis 1000 comprising a porous structure 1002 including plurality of transition zones. More particularly, the porous structure 1002 includes a first transition zone 1004 originating from a plane 1006 and extending at an angle to the longitudinal axis 1016 of the stem 1008, similar to the example of FIG. 27. The porous structure 1002 also includes a second transition zone 1010 originating from a plane 1012 and extending in a direction toward the medial aspect 1014 of the stem (e.g., along the x-axis in FIG. 28). The plane 1012 can be located between the longitudinal axis 1016 of the stem 1008 and the lateral aspect 1018 of the stem, such as at the transition between the surface of the lateral aspect 1018 and the cranial surface 1020. In certain examples, the porous structure 1002 can extend at least partially around the proximal portion 1022 of a longitudinally groove or recess 1024 formed in the stem 1008. Stated differently, the porous structure 1002 can extend distally and at least partially around the groove 1024. The porous structure 1002 (e.g., the low friction zone) can also extend at least partially inside the groove 1024. In other examples, the groove can be fully distal of the porous structure as in FIG. 27, depending upon the particular characteristics sought. In certain examples, the porous structure 1002 can have zones of exhibiting different coefficients of friction with bone, such low friction zones (e.g., the transition zones 1004 and 1010), moderate friction zones, and/or high friction zones. The zones can be longitudinally arranged on the stem similar to FIG. 27, or in other arrangements. In the example illustrated in FIG. 28, the high friction zone of the porous structure does not extend completely around the circumference of the stem, but it should be understood that in other examples the high friction zone can extend completely around the stem.

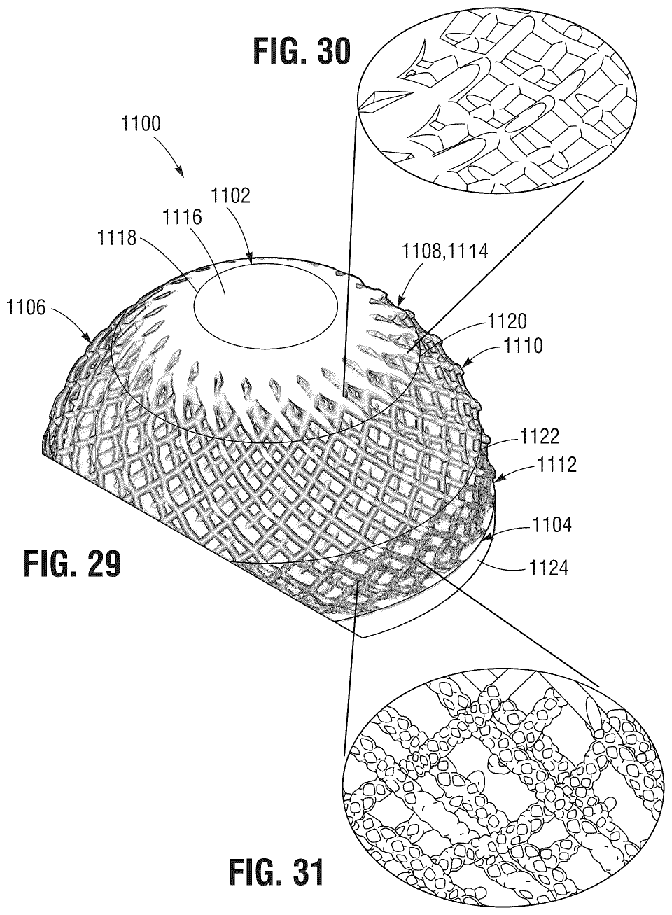

In certain examples, the porous lattice structures described herein can be shaped and sized to conform to the shape or outer profile of a particular implant. For example, FIG. 29 illustrates an example of a hemispherical acetabular cup 1100 including a first end portion 1102 encompassing the pole of the cup and a second end portion 1104 including the equator of the cup. The cup can include a porous lattice structure 1106 formed on the exterior of the cup. The lattice structure 1106 can comprise a plurality of interconnected struts, at least some of which extend in a curvilinear manner between the first end portion 1102 and the second end portion 1104 to conform to the spherical shape of the cup, referred to herein as a conformal lattice.

The porous structure 1106 can comprise a plurality of zones exhibiting different coefficients of friction with bone, as described previously. For example, the porous structure 1106 can comprise a first, relatively low friction zone 1108 at the upper latitudes of the cup, a second, moderate friction zone 1110 at the middle latitudes of the cup, and a third, relatively high friction zone 1112 at the equatorial latitudes of the cup. In the illustrated example, the low friction zone 1108 can include a transition region 1114 in which the struts decrease in diameter moving along their axes from the pole toward the equator of the cup. In certain examples, the low friction zone 1108 can include a cap or region 1116 of solid material at the pole of the cup. The transition zone 1114 can begin at the circumferential edge 1118 of the cap 1116 and can extend in a direction toward the equator. FIG. 30 is a magnified view of the struts in an exemplary portion of the low friction zone 1108.

A latitudinal line 1120 can indicate the end of the low friction zone 1108 and the start of the moderate friction zone 1110. Struts in the moderate friction zone 1110 can be shaped and sized similarly to those describe above with reference to FIG. 24.

A latitudinal line 1122 can indicate the end of the moderate friction zone 1110 and the start of the high friction zone 1112. Struts in the high friction zone 1112 can be shaped and sized similar to those described above with reference to FIG. 25. For example, struts in the high friction zone 1110 can comprise a Voronoi deboss surface structure of prominences and recesses as described above, or any other surface roughening treatment described herein. FIG. 31 is a magnified view of the struts in an exemplary portion of the high friction zone 1112.

In the illustrated embodiment, the second end portion 1104 can include a circumferential rim or ring 1124 of solid material at the equator of the cup, although other configurations are possible. The rim 1124 can form the lower or distal circumferential boundary of the high friction zone 1112.

In certain examples, the various zones of the porous structure 1106 can extend along portions of the overall height of the acetabular cup similar to the embodiment of FIG. 4 described above. For example, in certain embodiments the low friction zone 1108 can be 5% to 50% of the overall height of the acetabular cup as measured along the longitudinal axis, such as 5% to 40%, 5% to 30%, 5% to 20%, 5% to 10%, 10% to 50%, 10% to 40%, 10% to 30%, 10% to 20%, 20% to 50%, 20% to 40%, 20% to 30%, 30% to 50%, 30% to 40%, etc., of the overall height of the cup as measured along the longitudinal axis (see FIG. 4).

In certain examples, the moderate friction zone 1110 can be 5% to 60% of the overall height of the acetabular cup as measured along the longitudinal axis, such as 5% to 50%, 5% to 40%, 5% to 30%, 5% to 20%, 5% to 10%, 10% to 60%, 10% to 50%, 10% to 40%, 10% to 30%, 10% to 20%, 20% to 60%, 20% to 50%, 20% to 40%, 20% to 30%, 30% to 60%, 30% to 50%, 30% to 40%, 40% to 60%, etc., of the overall height of the cup as measured along the longitudinal axis (see FIG. 4).

In certain examples, the high friction zone 1112 can be 5% to 50% of the overall height of the acetabular cup as measured along the longitudinal axis, such as 5% to 40%, 5% to 30%, 5% to 20%, 5% to 10%, 10% to 50%, 10% to 40%, 10% to 30%, 10% to 20%, 20% to 50%, 20% to 40%, 20% to 30%, 30% to 50%, 30% to 40%, etc., of the overall height of the cup as measured along the longitudinal axis (see FIG. 4).

In certain examples, the porous lattice structures above can be formed using any of a variety of additive manufacturing processes, such as direct metal laser sintering, electron beam melting, etc.

### Additional Examples of the Disclosed Technology

In view of the above described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. An orthopedic implant, comprising: an implant body defining a longitudinal axis extending in a direction of implantation of the orthopedic implant, the implant body having a first end portion and a second end portion; and a porous structure extending circumferentially around the implant body, the porous structure comprising a first circumferentially-extending zone exhibiting a first coefficient of friction with bone tissue and a second circumferentially-extending zone exhibiting a second coefficient of friction with bone tissue, wherein the second coefficient of friction is greater than the first coefficient of friction; and wherein the first circumferentially-extending zone of the porous structure is offset from the second circumferentially-extending zone along the longitudinal axis such that during implantation of the orthopedic implant, the first circumferentially-extending zone of the porous structure contacts bone before the second circumferentially-extending zone of the porous structure.

Example 2. The orthopedic implant of any example herein, particularly example 1, wherein: the porous structure comprises a plurality of struts arranged in a three-dimensional arrangement; and the first circumferentially-extending zone comprises a transition zone in which a diameter of the struts decreases from a first diameter to a second diameter that is less than the first diameter.

Example 3. The orthopedic implant of any example herein, particularly example 2, wherein the diameter of the struts decreases in a direction that is at an angle to the longitudinal axis of the implant body.

Example 4. The orthopedic implant of any example herein, particularly example 2 or example 3, wherein the porous structure further comprises a second transition zone, and a diameter of struts in the second transition zone decreases in a different direction than in the first transition zone.

Example 5. The orthopedic implant of any example herein, particularly any one of examples 1-4, wherein struts of the second circumferentially-extending zone comprise a specified diameter along their lengths.

Example 6. The orthopedic implant of any example herein, particularly any one of examples 1-5, wherein the porous structure further comprises a third circumferentially-extending zone exhibiting a third coefficient of friction with bone that is higher than the first coefficient of friction and higher than the second coefficient of friction.

Example 7. The orthopedic implant of any example herein, particularly example 6, wherein struts of the third circumferentially-extending zone comprise a plurality of prominences and recesses that increase the third coefficient of friction.

Example 8. The orthopedic implant of any example herein, particularly any one of examples 1-7, wherein strut members of the first circumferentially-extending zone and/or the second circumferentially-extending zone comprise rounded end portions.

Example 9. The orthopedic implant of any example herein, particularly any one of examples 6-8, wherein end portions of the strut members of the third circumferentially-extending zone are pointed.

Example 10. The orthopedic implant of any example herein, particularly any one of examples 6-9, wherein strut members of the second circumferentially-extending zone and/or the third circumferentially-extending zone extend radially outwardly beyond strut members of the first circumferentially-extending zone.

Example 11. The orthopedic implant of any example herein, particularly any one of examples 1-10, wherein: the first circumferentially-extending zone of the porous structure exhibits a coefficient of friction with cancellous bone tissue of 0.2 to 0.5; and the second circumferentially-extending zone of the porous structure exhibits a coefficient of friction with cancellous bone tissue of 0.4 to 0.6.

Example 12. The orthopedic implant of any example herein, particularly any one of examples 6-11, wherein the third circumferentially-extending zone of the porous structure exhibits a coefficient of friction with cancellous bone tissue of 0.7 to 1.1.

Example 13. The orthopedic implant of any example herein, particularly any one of examples 6-12, wherein the orthopedic implant is an acetabular cup, and the third circumferentially-extending zone of the porous structure is disposed at least partially on the second end portion.

Example 14. The orthopedic implant of any example herein, particularly example 13, wherein a height of the third circumferentially-extending zone of the porous structure measured along the longitudinal axis is 10% to 40% of an overall height of the acetabular cup measured along the longitudinal axis.

Example 15. The orthopedic implant of any example herein, particularly any one of examples 6-12, wherein the orthopedic implant is a femoral stem prosthesis, and the third circumferentially-extending zone of the porous structure is at a proximal end of the porous structure.

Example 16. The orthopedic implant of any example herein, particularly example 15, wherein a height of the third circumferentially-extending zone of the porous structure measured in a direction of the strut diameter decrease in the transition zone is 10% to 40% of an overall height of the porous structure measured in the direction of the strut diameter decrease in the transition zone.

Example 17. An orthopedic implant, comprising: an implant body defining a longitudinal axis extending in a direction of implantation of the orthopedic implant, the implant body having a first end portion and a second end portion; the implant body having a thickness dimension defined perpendicular to the longitudinal axis, the thickness dimension of the implant body increasing in a direction along the longitudinal axis from the first end portion to the second end portion; and the implant body comprising a porous structure extending along the longitudinal axis, the porous structure comprising a plurality of struts arranged in a three-dimensional arrangement, the porous structure comprising a transition zone in which the porous structure gradually develops from a solid bulk material of the implant body over a selected distance.

Example 18. The orthopedic implant of any example herein, particularly example 17, wherein the porous structure exhibits a coefficient of friction with bone tissue that decreases along the porous structure in a direction of implantation of the orthopedic implant.

Example 19. The orthopedic implant of any example herein, particularly example 17 or example 18, wherein a diameter of the struts of the transition zone decreases in a direction that is at an angle to the longitudinal axis of the implant body.

Example 20. The orthopedic implant of any example herein, particularly example 17 or example 18, wherein: the transition zone is a first zone of the porous structure and exhibits a first coefficient of friction with bone; and the porous structure further comprises a second zone and a third zone, the second zone being offset from the first zone in the direction of implantation and the third zone being offset from the second zone in the direction of implantation such that the second zone is between the first zone and the third zone, and such that the first zone contacts bone before the second zone of the porous structure during implantation; wherein the third zone exhibits a coefficient of friction with bone tissue that is greater than a coefficient of friction with bone tissue exhibited by the second zone.

Example 21. The orthopedic implant of any example herein, particularly example 20, wherein struts of the second zone comprise a specified diameter along their lengths.

Example 22. The orthopedic implant of any example herein, particularly any one of examples 20-21, wherein struts of the third zone comprise a plurality of prominences and recesses that increase the third coefficient of friction.

Example 23. The orthopedic implant of any example herein, particularly any one of examples 20-22, wherein: the first zone of the porous structure exhibits a coefficient of friction with cancellous bone tissue of 0.2 to 0.5; the second zone of the porous structure exhibits a coefficient of friction with cancellous bone tissue of 0.4 to 0.6; and the third zone of the porous structure exhibits a coefficient of friction with cancellous bone tissue of 0.7 to 1.1.

Example 24. The orthopedic implant of any example herein, particularly any one of examples 20-23, wherein the first zone, the second zone, and the third zone of the porous structure each extend at least partially around a perimeter of the implant body.

### Explanation of Terms

For purposes of this description, certain aspects, advantages, and novel features of the embodiments of this disclosure are described herein. The disclosed methods, apparatus, and systems should not be construed as being limiting in any way. Instead, the present disclosure is directed toward all novel and nonobvious features and aspects of the various disclosed embodiments, alone and in various combinations and sub-combinations with one another. The methods, apparatus, and systems are not limited to any specific aspect or feature or combination thereof, nor do the disclosed embodiments require that any one or more specific advantages be present or problems be solved.

Although the operations of some of the disclosed embodiments are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods.

As used in this disclosure and in the claims, the singular forms “a,” “an,” and “the” include the plural forms unless the context clearly dictates otherwise. Additionally, the term “includes” means “comprises.” Further, the terms “coupled” and “associated” generally mean electrically, electromagnetically, and/or physically (e.g., mechanically or chemically) coupled or linked and does not exclude the presence of intermediate elements between the coupled or associated items absent specific contrary language.

In some examples, values, procedures, or apparatus may be referred to as “lowest,” “best,” “minimum,” or the like. It will be appreciated that such descriptions are intended to indicate that a selection among many alternatives can be made, and such selections need not be better, smaller, or otherwise preferable to other selections.

In the description, certain terms may be used such as “up,” “down,” “upper,” “lower,” “horizontal,” “vertical,” “left,” “right,” and the like. These terms are used, where applicable, to provide some clarity of description when dealing with relative relationships. But, these terms are not intended to imply absolute relationships, positions, and/or orientations. For example, with respect to an object, an “upper” surface can become a “lower” surface simply by turning the object over. Nevertheless, it is still the same object.

Unless otherwise indicated, all numbers expressing angles, dimensions, quantities of components, forces, moments, molecular weights, percentages, times, and so forth, as used in the specification or claims are to be understood as being modified by the term “about.” Accordingly, unless otherwise indicated, implicitly or explicitly, the numerical parameters set forth are approximations that can depend on the desired properties sought and/or limits of detection under test conditions/methods familiar to those of ordinary skill in the art. When directly and explicitly distinguishing embodiments from discussed prior art, the embodiment numbers are not approximates unless the word “about” is recited.

Although there are alternatives for various components, parameters, operating conditions, etc., set forth herein, that does not mean that those alternatives are necessarily equivalent and/or perform equally well. Nor does it mean that the alternatives are listed in a preferred order unless stated otherwise.

As used herein, values or terms modified by the term “substantially” mean±10% of the stated value.

In view of the many possible embodiments to which the principles of the disclosed technology may be applied, it should be recognized that the illustrated embodiments are only examples and should not be taken as limiting the scope of the disclosure. Rather, the scope of the disclosure is at least as broad as the following claims and their equivalents. We therefore claim as all that comes within the scope and spirit of these claims.

1. An orthopedic implant, comprising:
an implant body defining a longitudinal axis extending in a direction of implantation of the orthopedic implant, the implant body having a first end portion and a second end portion; and
a porous structure extending circumferentially around the implant body, the porous structure comprising a first circumferentially-extending zone exhibiting a first coefficient of friction with bone tissue and a second circumferentially-extending zone exhibiting a second coefficient of friction with bone tissue, wherein the second coefficient of friction is greater than the first coefficient of friction; and
wherein the first circumferentially-extending zone of the porous structure is offset from the second circumferentially-extending zone along the longitudinal axis such that during implantation of the orthopedic implant, the first circumferentially-extending zone of the porous structure contacts bone before the second circumferentially-extending zone of the porous structure.

2. The orthopedic implant of claim 1, wherein:
the porous structure comprises a plurality of struts arranged in a three-dimensional arrangement; and
the first circumferentially-extending zone comprises a transition zone in which a diameter of the struts decreases from a first diameter to a second diameter that is less than the first diameter.

3. The orthopedic implant of claim 2, wherein the diameter of the struts decreases in a direction that is at an angle to the longitudinal axis of the implant body.

4. The orthopedic implant of claim 2, wherein:
the transition zone of the first circumferentially-extending zone is a first transition zone; and
the porous structure further comprises a second transition zone, and a diameter of struts in the second transition zone decreases in a different direction than in the first transition zone.

5. The orthopedic implant of claim 1, wherein struts of the second circumferentially-extending zone comprise a specified diameter along their lengths.

6. The orthopedic implant of claim 2, wherein the porous structure further comprises a third circumferentially-extending zone exhibiting a third coefficient of friction with bone that is higher than the first coefficient of friction and higher than the second coefficient of friction.

7. The orthopedic implant of claim 6, wherein struts of the third circumferentially-extending zone comprise a plurality of prominences and recesses that increase the third coefficient of friction.

8. The orthopedic implant of claim 1, wherein struts of the first circumferentially-extending zone and/or the second circumferentially-extending zone comprise rounded end portions.

9. The orthopedic implant of claim 6, wherein end portions of struts of the third circumferentially-extending zone are pointed.

10. The orthopedic implant of claim 6, wherein strut members of the second circumferentially-extending zone and/or the third circumferentially-extending zone extend radially outwardly beyond strut members of the first circumferentially-extending zone.

11. The orthopedic implant of claim 1, wherein:
the first circumferentially-extending zone of the porous structure exhibits a coefficient of friction with cancellous bone tissue of 0.2 to 0.5; and
the second circumferentially-extending zone of the porous structure exhibits a coefficient of friction with cancellous bone tissue of 0.4 to 0.6.

12. The orthopedic implant of claim 6, wherein the third circumferentially-extending zone of the porous structure exhibits a coefficient of friction with cancellous bone tissue of 0.7 to 1.1.

13. The orthopedic implant of claim 6, wherein the orthopedic implant is an acetabular cup, and the third circumferentially-extending zone of the porous structure is disposed at least partially on the second end portion.

14. The orthopedic implant of claim 13, wherein a height of the third circumferentially-extending zone of the porous structure measured along the longitudinal axis is 10% to 40% of an overall height of the acetabular cup measured along the longitudinal axis.

15. The orthopedic implant of claim 6, wherein the orthopedic implant is a femoral stem prosthesis, and the third circumferentially-extending zone of the porous structure is at a proximal end of the porous structure.

16. The orthopedic implant of claim 15, wherein a height of the third circumferentially-extending zone of the porous structure measured in a direction of the strut diameter decrease in the transition zone is 10% to 40% of an overall height of the porous structure measured in the direction of the strut diameter decrease in the transition zone.

17. An orthopedic implant, comprising:
an implant body defining a longitudinal axis extending in a direction of implantation of the orthopedic implant, the implant body having a first end portion and a second end portion;
the implant body having a thickness dimension defined perpendicular to the longitudinal axis, the thickness dimension of the implant body increasing in a direction along the longitudinal axis from the first end portion to the second end portion; and
the implant body comprising a porous structure extending along the longitudinal axis, the porous structure comprising a plurality of struts arranged in a three-dimensional arrangement, the porous structure comprising a transition zone in which the porous structure gradually develops from a solid bulk material of the implant body over a selected distance.

18. The orthopedic implant of claim 17, wherein the porous structure exhibits a coefficient of friction with bone tissue that decreases along the porous structure in a direction of implantation of the orthopedic implant.

19. The orthopedic implant of claim 17, wherein a diameter of the struts of the transition zone decreases in a direction that is at an angle to the longitudinal axis of the implant body.

20. The orthopedic implant of claim 17, wherein:
the transition zone is a first zone of the porous structure and exhibits a first coefficient of friction with bone; and
the porous structure further comprises a second zone and a third zone, the second zone being offset from the first zone in the direction of implantation and the third zone being offset from the second zone in the direction of implantation such that the second zone is between the first zone and the third zone, and such that the first zone contacts bone before the second zone of the porous structure during implantation;
wherein the third zone exhibits a third coefficient of friction with bone tissue that is greater than a second coefficient of friction with bone tissue exhibited by the second zone.

21. The orthopedic implant of claim 20, wherein struts of the second zone comprise a specified diameter along their lengths.

22. The orthopedic implant of claim 20, wherein struts of the third zone comprise a plurality of prominences and recesses that increase the third coefficient of friction.

23. The orthopedic implant of claim 20, wherein:
the first zone of the porous structure exhibits a coefficient of friction with cancellous bone tissue of 0.2 to 0.5;
the second zone of the porous structure exhibits a coefficient of friction with cancellous bone tissue of 0.4 to 0.6; and
the third zone of the porous structure exhibits a coefficient of friction with cancellous bone tissue of 0.7 to 1.1.

24. The orthopedic implant of claim 20, wherein the first zone, the second zone, and the third zone of the porous structure each extend at least partially around a perimeter of the implant body.
